(19) United States
(12) Patent Application Publication
(10) Pub. No.: US 2024/0003907 A1
(43) Pub. Date: Jan. 4, 2024

(54) METHODS OF ASSESSING RISK OF AND TREATING PREECLAMPSIA AND SUBTYPES THEREOF

(71) Applicants: NX Prenatal Inc., Louisville, KY (US); The Brigham and Women's Hospital, Inc., Boston, MA (US)

(72) Inventors: Kevin P. ROSENBLATT, Louisville, KY (US); Thomas F. MCELRATH, Boston, MA (US)

(21) Appl. No.: 18/096,464

(22) Filed: Jan. 12, 2023

Related U.S. Application Data
(63) Continuation of application No. PCT/US2021/041326, filed on Jul. 12, 2021.
(60) Provisional application No. 63/051,371, filed on Jul. 13, 2020.

(51) Int. Cl. G01N 33/68; G01N 33/86

(52) U.S. Cl. CPC G01N 33/689; G01N 33/86; G01N 2800/368; G01N 2333/4716; G01N 2333/78; G01N 2333/75; G01N 2333/96458; G01N 2333/4737; G01N 2800/50

(57) ABSTRACT

Disclosed herein are CMP-associated proteins, collected between 10-12 weeks of gestation, that can be used to stratify the risk of later preeclampsia requiring delivery at <=35 weeks of gestation. For those that screen positive, clustering can be used to further characterize the putative subtype of the preeclampsia. Risk stratification and disease phenotype characterizations such as this can optimize prophylactic and therapeutic interventions.

### CROSS REFERENCE TO RELATED APPLICATIONS

This application claims priority to U.S. provisional patent application number 63/051,371, filed on Jul. 13, 2020, the content of which is incorporated herein by reference in its entirety.

### BACKGROUND

Preeclampsia is a human affliction with no naturally occurring mammalian models. This observation suggests that the origins of the condition lie in the unique aspects of human placentation and gestational adaptation. It is believed that rather than it being a single disease entity, it is more likely to be a syndrome with a core set of common features, with multiple associated subtypes.

Conventionally, only early versus late-onset distinctions of preeclampsia have been elucidated. Needed are an understanding of patterns that indicate risk of subphenotypes of preeclampsia beyond the conventional early versus late-onset distinction, allowing for treatment/prevention strategies that would address the subtype or stage of preeclampsia for which the patient is at risk. Accordingly provided herein are methods, models, and compositions that address this need.

### SUMMARY

In one aspect provided herein is a method for determining a subtype of preeclampsia in a pregnant subject, the method comprising: (a) preparing a microparticle-enriched fraction from a blood sample from a pregnant subject at increased risk of preeclampsia; (b) determining a measure of one or more microparticle-associated protein biomarkers in the fraction, wherein protein biomarkers include at least one coagulation-related biomarker and/or at least one complement activity-related biomarker; and (c) assessing the form of preeclampsia based on the measure, wherein: altered expression of a coagulation-related biomarker indicates blood coagulation-associated preeclampsia; and altered expression of a complement activity-related biomarker indicates complement-type preeclampsia. In one embodiment the method further comprises determining that the subject is at increased risk of preeclampsia: (a) determining a measure of one or more microparticle-associated protein biomarkers in a microparticle-enriched fraction from the subject, wherein the one or more protein biomarkers are associated with increased risk of preeclampsia; and (c) determining an increased risk of the risk of preeclampsia, based on the measure or measures. In another embodiment the biomarkers are selected from: (i) a protein biomarker of Table 2; and (ii) a protein biomarker of Table 3. In another embodiment the panel comprises (1) complement C1r subcomponent protein (C1RL), platelet glycoprotein 1 b alpha chain (GP1BA), vitronectin (VTNC), and zinc-alpha-2-glycoprotein (ZA2G) or (2) complement C1r subcomponent protein (C1RL), platelet glycoprotein 1 b alpha chain (GP1 BA), vitronectin (VTNC), and beta-2-glycoprotein 1 (APOH). In another embodiment: (i) one or a plurality (e.g., at least any of two, three, four or five) of the coagulation-related biomarkers is selected from the group consisting of Immunoglobulin J chain, Zinc finger protein 251, Extracellular matrix protein 1, CD5 antigen-like, and Alpha-2-macroglobulin; and (b) one or a plurality (e.g., at least any of two, three, four or five six, seven, eight, nine, ten, eleven, twelve, thirteen, fourteen, fifteen, sixteen or seventeen) of the complement activity-related biomarkers is selected from the group consisting of Vitronectin, Pigment epithelium-derived factor, Complement C4-A, Prothrombin, Highly similar to Complement factor B, Complement C3, Angiotensinogen, Complement C2, Phosphatidylinositol-glycan-specific phospholipase D, Coagulation factor XII, Complement factor H, Calcium-dependent secretion activator 1, C-reactive protein, Kininogen-1, Heparin cofactor 2, Hemopexin, and Bone marrow proteoglycan. In another embodiment the altered expression associated with coagulation-associated preeclampsia is an increased expression compared with normal, and/or the altered expression associated with complement activity-associated preeclampsia is an increased expression compared with normal.

In another aspect provided herein is a method of treating a subtype of preeclampsia in a pregnant subject comprising: (a) determining whether the subject is at increased risk of coagulation-associated preeclampsia (e.g. associated with platelet activation) or complement activity-type preeclampsia, wherein determining comprises associating a measure of one or more protein coagulation-related biomarkers or one or more complement activity-related biomarkers with coagulation-associated preeclampsia or complement activity-type preeclampsia; and (b) treating the subject as follows: (1) if the subject is determined to be at increased risk of coagulation-associated preeclampsia, administering to the subject a therapeutic intervention to decrease abnormal blood coagulation; or (2) if the subject is determined to be at increased risk of complement activity-type preeclampsia, administering to the subject a therapeutic intervention to decrease abnormal complement activity. In another embodiment the method comprises, before determining the type of preeclampsia, determining that the subject is at increased risk for preeclampsia requiring. In another embodiment the therapeutic intervention for coagulation-associated preeclampsia, comprises administration of a platelet aggregation inhibitor (e.g., aspirin, clopidogrel, cilostazol, prasugrel, ticagrelor, caplacizyumab). In another embodiment the therapeutic intervention for complement activity-type type preeclampsia, comprises administration of a pharmaceutical selected from a protease inhibitor (e.g., an inhibitor of C1r/C1s, kallikrein, C1 inhibitor, serine protease inhibitor), a soluble complement regulator (e.g., factor I cofactor), an anti-complement antibody (e.g., anti-C5, anti-C5a, anti-CD20, anti-CD38), anti-Factor D, anti-factor B, anti-properdin), a complement component inhibitor, a receptor antagonist.

In another aspect provided herein is a method for assessing risk of preeclampsia requiring delivery in <=35 weeks gestation, in a pregnant subject, the method comprising: (a) preparing a microparticle-enriched fraction from a blood sample from the pregnant subject; (b) determining a measure of one or more microparticle-associated protein biomarkers in the fraction, wherein the one or more protein biomarkers are selected from: (i) a protein biomarker of Table 2; and (ii) a protein biomarker of Table 3; and (c) assessing the risk of preeclampsia requiring delivery at <=35 weeks gestation based on the measure. In another embodiment assessing the risk comprises distinguishing risk of preeclampsia requiring delivery at <=35 weeks gestation and preeclampsia not requiring delivery at <=35 weeks gestation. In another embodiment an increased amount of an up-regulated biomarker or a decreased amount of a down-regulated biomarker indicates increased risk of preeclampsia requiring delivery in <=35 weeks gestation. In another embodiment the method comprises determining a measure of one or a plurality of protein biomarkers selected from the protein biomarkers of Table 2. In another embodiment the biomarkers comprise a panel of at least four biomarkers, wherein one or a plurality of the biomarkers in the panel are selected from the biomarkers of Table 2. In another embodiment the panel comprises one, two, three, four, five, six or seven biomarkers selected from GP1BA, VTNC, C1RL, ZA2G, APOC2, APOH, and JPH1. In another embodiment the panel comprises complement C1r subcomponent protein (C1RL), platelet glycoprotein 1b alpha chain (GP1BA), vitronectin (VTNC), and zinc-alpha-2-glycoprotein (ZA2G). In another embodiment the panel comprises complement C1r subcomponent protein (C1RL), platelet glycoprotein 1b alpha chain (GP1BA), vitronectin (VTNC), and beta-2-glycoprotein 1 (APOH). In another embodiment the panel comprises no more than any of 10, 9, 8, 7, 6, 5, or 4 protein biomarkers. In another embodiment the sample is taken from the pregnant subject during the first trimester or second trimester of pregnancy. In another embodiment wherein the sample is taken from the pregnant subject during weeks 10-12 of gestation. In another embodiment the pregnant subject is primigravida, multigravida, primiparous or multiparous. In another embodiment the pregnant subject has a singleton pregnancy or multiple pregnancy. In another embodiment the pregnant subject is asymptomatic for preeclampsia, e.g., is not hypertensive or does not have proteinuria. In another embodiment the pregnant subject has no history of preeclampsia. In another embodiment the pregnant subject has no other risk factors for preeclampsia. In another embodiment the pregnant subject has chronic hypertension. In another embodiment the blood sample is plasma or serum. In another embodiment the microparticle-enriched fraction is prepared using size-exclusion chromatography. In another embodiment the size-exclusion chromatography comprises elution with water. In another embodiment the size-exclusion chromatography is performed with an agarose solid phase and an aqueous liquid phase. In another embodiment the preparing step further comprises using ultrafiltration or reverse-phase chromatography. In another embodiment the preparing step further comprises denaturation using urea, reduction using dithiothreitol, alkylation using iodoacetamine, and digestion using trypsin after the size exclusion chromatography. In another embodiment the microparticles are further purified to enrich for placental-derived exosomes or vascular endothelial-derived exosomes. In another embodiment determining a measure comprises mass spectrometry. In another embodiment determining a measure comprises liquid chromatography/mass spectrometry (LC/MS). In another embodiment mass spectrometry comprises liquid chromatography/triple quadrupole mass spectrometry. In another embodiment the mass spectrometry comprises multiple reaction monitoring. In another embodiment the mass spectrometry comprises multiple reaction monitoring, and the liquid chromatography is done using a solvent comprising acetonitrile, and/or determining comprises assigning an indexed retention time to the protein biomarkers. In another embodiment the mass spectrometry comprises multiple reaction monitoring, and the method comprises adding one or more stable isotope standard peptides to the sample before introduction into the mass spectrometer and detection comprises detecting one or a plurality of daughter ions of the stable isotope peptide standards produced by a collision cell of the mass spectrometer. In another embodiment determining the measure comprises determining a measure of a surrogate peptide of the protein biomarker. In another embodiment mass spectrometry comprises quantifying one or more stable isotope labeled standard peptides (SIS peptides) corresponding to each of the surrogate peptides. In another embodiment the method comprises adding one or more stable heavy isotope substituted standards corresponding to said protein biomarkers to the microparticle enriched fraction. In another embodiment determining a measure comprises contacting the sample with one or more capture reagents, each capture reagent specifically binding one of the protein biomarkers, and detecting binding between the capture reagent in the protein biomarker. In another embodiment the method comprises performing an immunoassay. In another embodiment the immunoassay is selected from the group consisting of enzyme immunoassay (EIA), enzyme-linked immunosorbent assay (ELISA), and radioimmunoassay (RIA). In another embodiment the assessing comprises executing a classification rule, which rule classifies the subject as being at increased risk of preeclampsia requiring delivery in <=35 weeks gestation, as being at increased risk of blood coagulation-associated preeclampsia and/or as being at increased risk of complement-type preeclampsia, and wherein execution of the classification rule produces a correlation between preeclampsia requiring delivery in <=35 weeks gestation or term birth with a p value of less than at least 0.05. In another embodiment the assessing comprises executing a classification rule, which rule classifies the subject at being at risk of preeclampsia, and wherein execution of the classification rule produces a receiver operating characteristic (ROC) curve, wherein the ROC curve has an area under the curve (AUC) of at least 0.6, at least 0.7, at least 0.8 or at least 0.9. In another embodiment values on which the classification rule classifies a subject further include at least one of: maternal age, maternal body mass index, primiparous, and smoking during pregnancy. In another embodiment the classification rule employs cut-off, linear regression (e.g., multiple linear regression (MLR), partial least squares (PLS) regression, principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (e.g., support vector machines). In another embodiment the classification rule is configured to have a sensitivity, specificity, positive predictive value or negative predictive value of at least 70%, least 80%, at least 90% or at least 95%. In another embodiment assessing an increased risk of preeclampsia comprises determining that the protein biomarker (if upregulated) is above or (if down regulated) is below a threshold level. In another embodiment the threshold level represents a level at least one, at least two or at least three z scores from a measure of central tendency (e.g., mean, median or mode) for the protein determined from at least 50, at least 100 or at least 200 control subjects. In another embodiment the assessing comprises comparing the measure of each protein in the panel to a reference standard. In another embodiment the method further comprises communicating the risk of preeclampsia for a pregnant subject to a health care provider. In another embodiment the method further comprises: (d) determining, a measure of one or more microparticle-associated protein biomarkers for preterm birth in the fraction; and (e) assessing the risk of preterm birth based on the measure.

In another aspect provided herein is a method of decreasing risk of preeclampsia requiring delivery in <=35 weeks gestation in a pregnant subject and/or reducing neonatal complications of preeclampsia requiring delivery in <=35 weeks gestation, the method comprising: (a) assessing risk of preeclampsia requiring delivery in <=35 weeks gestation for a pregnant subject; and (b) for a subject determined to be at increased risk of preeclampsia requiring delivery in <=35 weeks gestation, administering a therapeutic intervention to the subject effective to decrease the risk of preeclampsia and/or reduce neonatal complications of preeclampsia. In another embodiment the therapeutic intervention comprises administration of a platelet aggregation inhibitor or a complement activity inhibitor. In another embodiment the therapeutic intervention is selected from the group consisting of aspirin (e.g., low dose aspirin), a corticosteroid or a medication to reduce hypertension.

In another aspect provided herein is a method comprising administering to a pregnant subject determined by a method as described herein to be at increased risk of a condition selected from blood coagulation-associated preeclampsia or complement-activity related preeclampsia, a therapeutic intervention effective to reduce the risk of said condition.

In another aspect provided herein is a method comprising administering to a pregnant subject having an altered measure as compared to a reference standard of any one of the protein biomarkers selected from Table 2, Table 3, or Table 4 an effective amount of a treatment designed to reduce the risk of preeclampsia, blood coagulation-associated preeclampsia or complement-activity related preeclampsia.

In another aspect provided herein is a composition comprising one or a plurality of pairs of polypeptides, each pair comprising a protein biomarkers or surrogate biomarkers selected from the protein biomarkers of Table 2, Table 3 or Table 4 and, for each biomarker, a stable isotope standard peptide corresponding to the biomarker.

In another aspect provided herein is a kit comprising one or a plurality of containers, wherein each container comprises one or more of each of a plurality of Stable Isotopic Standards, each stable isotopic standard corresponding to a surrogate peptide for a protein biomarker selected from the biomarkers of Table 2, Table 3, or Table 4.

In another aspect provided herein is a computer readable medium in tangible, non-transitory form comprising code to implement a classification rule generated by a method as described herein.

In another aspect provided herein is a system comprising: (a) a computer comprising: (i) a processor; and (II) a memory, coupled to the processor, the memory storing a module comprising: (1) test data for a sample from a subject including values indicating a measure of one or more protein biomarkers in the fraction, wherein the protein biomarkers are selected from the protein biomarkers of Table 2, Table 3 and Table 4; (2) a classification rule which, based on values including the measurements, classifies the subject as being at increased risk of preeclampsia requiring delivery in <=35 weeks gestation, blood coagulation-associated preeclampsia or complement-activity related preeclampsia, wherein the classification rule is configured to have a sensitivity of at least 75%, at least 85% or at least 95%; and (3) computer executable instructions for implementing the classification rule on the test data.

In another aspect provided herein is a computer-implemented method for generating a model to assess a risk of preeclampsia. The computer-implemented method can comprise obtaining a dataset. The dataset can comprise measurements associated with a plurality of biomarkers derived from each of a plurality of subjects. The computer-implemented method can also comprise implementing a machine learning analysis to associate a set of biomarkers within the plurality of biomarkers with preeclampsia. Implementing the machine learning analysis can generate a model to assess the risk of preeclampsia. The risk of preeclampsia can be one or more of risk of preeclampsia requiring delivery in <=35 weeks gestation, risk of blood coagulation-associated preeclampsia and risk of complement-activity related preeclampsia. The biomarkers are selected from the biomarkers of Table 2, Table 3, and Table 4.

In some aspects, assessing risk can comprise classifying a subject as being at one of increased risk or decreased risk of preeclampsia (e.g. risk of preeclampsia requiring delivery in <=35 weeks gestation, risk of blood coagulation-associated preeclampsia and/or risk of complement-activity related preeclampsia). In some aspects, assessing risk can comprise determining a likelihood of a subject developing preeclampsia (e.g. risk of developing preeclampsia requiring delivery in <=35 weeks gestation, risk of developing blood coagulation-associated preeclampsia and/or risk of developing complement-activity related preeclampsia). In some aspects, the model executes at least one classification rule to assess the risk of preeclampsia. The at least one classification rule can comprise at least one of cut-offs, linear regression, binary decision trees, artificial neural networks, discriminant analyses, logistic classifiers, and support vector classifiers.

In some aspects, the model executes at least one classification rule to assess the risk of preeclampsia. The at least one classification rule can produce a receiver operating characteristic (ROC) curve, and wherein the ROC curve has an area under the curve (AUC) of at least 0.6, at least 0.7, at least 0.8 or at least 0.9.

In another aspect, provided herein is a computer-implemented method of assessing a risk of preeclampsia in a subject. The computer-implemented method can comprise determining a quantitative measure of at least one biomarker in a sample, and executing a classification rule based on the quantitative measure. The execution of the classification rule can assess the risk of preeclampsia in the subject (e.g. risk of preeclampsia requiring delivery in <=35 weeks gestation, risk of blood coagulation-associated preeclampsia and/or risk of complement-activity related preeclampsia). The classification rule can implement at least one of cut-offs, linear regression, binary decision trees, artificial neural networks, discriminant analyses, logistic classifiers, and support vector classifiers. The at least one biomarker can be selected from the biomarkers of Table 2, Table 3, and Table 4.

In some aspects, the classification rule can produce a receiver operating characteristic (ROC) curve. The ROC curve has an area under the curve (AUC) of at least 0.6, at least 0.7, at least 0.8 or at least 0.9. In some aspects, the classification rule is configured to have a sensitivity of at least 75%, at least 85%, or at least 95%.

In another aspect, provided herein is a system to assess risk of preeclampsia in a subject. The system can comprise a processor, and a memory coupled to the processor. The memory can store: (i) a first dataset comprising a first plurality of measurements associated with a plurality of biomarkers derived from each of a plurality of subjects, (ii) a second dataset comprising a second plurality of measurements associated with the plurality of biomarkers derived from another subject, and (iii) computer-readable instructions to: (1) implement a machine learning analysis to associate a set of biomarkers within the plurality of biomarkers within the first dataset, wherein the machine learning analysis generates a model to assess the risk of preeclampsia, wherein the set of protein biomarkers are selected from the biomarkers of Table 2, Table 3, and Table 4; (2) execute a classification rule based on the second plurality of measurements from the other subject, wherein the execution of the classification rule assesses the risk of preeclampsia in the other subject. The risk of preeclampsia is a risk of preeclampsia requiring delivery in <=35 weeks gestation, risk of blood coagulation-associated preeclampsia and/or risk of complement-activity related preeclampsia.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1. Schematic of workflow for case vs. control CMP associated protein identification

FIG. 2. Circulating microparticle proteins associated with preeclampsia at median 12 weeks gestation. Boxed dots represent CMPs that were significantly associated with preeclampsia at an adjusted p-value<0.05. Black dots represent CMPs that were not significantly associated with preeclampsia at an adjusted p-value<0.05.

FIG. 3. AUC vs. standard deviation for protein vs. permuted. Blue dots represent actual protein AUC and SD. The dots with an “X” represent AUC and SD from randomly permuted the sample labels (preeclampsia versus control).

FIG. 4. Density plots of protein vs. permuted. Unhatched areas represent actual protein AUC and SD. The hatched areas represent AUC and SD from randomly permuted the sample labels (preeclampsia versus control).

FIG. 5. K-Means clustering of cases of preeclampsia delivering <=35 weeks gestation. Dots are circled representing observed cluster 1 of cases and cluster 2 of cases.

FIG. 6 shows a cluster of proteins identified herein as being predictive for coagulation-associated preeclampsia (e.g. associated with platelet activation) (Cluster 1).

FIG. 7 shows a cluster of proteins identified herein as being predictive for complement-associated preeclampsia (e.g. associated with complement activation) (Cluster 2).

### DETAILED DESCRIPTION

### I. Introduction

Disclosed herein are methods, compositions, systems and articles of manufacture useful in determining risk of developing, and for treating, preeclampsia, in particular, preeclampsia requiring delivery in <=35 weeks gestation, and for classifying women at risk of preeclampsia as having a subtype of preeclampsia, e.g., either coagulation-associated preeclampsia (e.g. associated with platelet activation) or complement-associated preeclampsia. Coagulation-associated preeclampsia is characterized by altered (e.g., elevated) expression of proteins that are involved in blood coagulation. Complement-associated preeclampsia is characterized by altered (e.g., elevated) expression of proteins involved in complement activity. Risk of developing preeclampsia requiring delivery in <=35 weeks gestation can be determined while the condition is sub-clinical and/or below normal threshold for detection. In some embodiments, determination involves detection of preeclampsia biomarkers found in microparticle-enriched fractions from the blood of pregnant women. Such biomarkers are presented in Table 2, Table 3 and Table 4.

### II. Subjects

Subjects for prediction and treatment of preeclampsia requiring delivery in <=35 weeks gestation are pregnant human females. In some embodiments, the pregnant woman is in the first trimester (e.g., weeks 1-12 of gestation), second trimester (e.g., weeks 13-28 of gestation) or third trimester (e.g., weeks 29-37 of gestation) of pregnancy. In some embodiments, the pregnant woman is in early pregnancy (e.g., from 8, 9, 10, 11, 12, 13, 14, 15, 16, 17, 18, 19 or 20, but earlier than 21 weeks of gestation; from 20, 19, 18, 17, 16, 15, 14, 13, 12, 11, 10 or 9, but later than 8 weeks of gestation). In some embodiments, the pregnant woman is between 8-15 weeks of pregnancy, for example, 10-12 weeks, 8-12 weeks or 10-15 weeks. In some embodiments, the pregnant woman is in mid-pregnancy (e.g., from 21, 22, 23, 24, 25, 26, 27, 28, 29 or 30, but earlier than 31 weeks of gestation; from 30, 29, 28, 27, 26, 25, 24, 23, 22 or 21, but later than 20 weeks of gestation). In some embodiments, the pregnant woman is in late pregnancy (e.g., from 31, 32, 33, 34, 35, 36 or 37, but earlier than 38 weeks of gestation; from 37, 36, 35, 34, 33, 32 or 31, but later than 30 weeks of gestation). In some embodiments, the pregnant woman is in less than 17 weeks, less than 16 weeks, less than 15 weeks, less than 14 weeks or less than 13 weeks of gestation; from 20, 19, 18, 17, 16, 15, 14, 13, 12, 11, 10 or 9, but later than 8 weeks of gestation). The stage of pregnancy can be calculated from the first day of the last normal menstrual period of the pregnant subject.

Pregnant subjects of the methods described herein can belong to one or more classes including primiparous (no previous child brought to delivery, interchangeably referred to herein as nulliparous or parity=0) or multiparous (at least one previous child brought to at least 20 weeks of gestation, referred to interchangeably herein as parity>0, parity≥1), primigravida (first pregnancy) or multigravida (more than one pregnancy).

In some embodiments, the pregnant human subject is asymptomatic. In some embodiments, the subject may have a risk factor of preeclampsia such as high blood pressure, protein in the urine, a family history of preeclampsia, renal or connective tissue disease, obesity, advanced maternal age, or a conception with medical assistance.

### III. Sample Preparation

A sample for use in the methods of the present disclosure is a biological sample obtained from a pregnant subject. In certain embodiments, the sample is collected during a stage of pregnancy described in the preceding section. In some embodiments, the sample is a blood, saliva, tears, sweat, nasal secretions, urine, amniotic fluid or cervicovaginal fluid sample. In some embodiments, the sample is a blood sample, which in certain embodiments are serum or plasma. In some embodiments, the sample has been stored frozen (e.g., −20° C. or −80° C.).

The term “microparticle” refers to an extracellular microvesicle or lipid raft protein aggregate having a hydrodynamic diameter of about 50 to about 5000 nm. As such, the term microparticle encompasses exosomes (about 50 to about 100 nm), microvesicles (about 100 to about 300 nm), ectosomes (about 50 to about 1000 nm), apoptotic bodies (about 50 to about 5000 nm) and lipid-protein aggregates of the same dimensions.

The term “microparticle-associated protein” refers to a protein or fragment thereof that is detectable in a microparticle-enriched sample from a mammalian (e.g., human) subject. As such the term “microparticle-associated protein” is not restricted to proteins or fragments thereof that are physically associated with microparticles at the time of detection.

The term “polypeptide” as used herein refers to a polymer of amino acids. This includes oligopeptides, which typically have fewer than 10 amino acids, peptides, which typically have between about 10 and about 50 amino acids, and proteins, which include polypeptides assuming secondary, tertiary or quaternary structures. Depending on context, the term “protein” may refer to a polypeptide lacking secondary structure.

The term “about” as used herein in reference to a value refers to 90% to 110% of that value. For instance, a diameter of about 1000 nm is a diameter within the range of 900 nm to 1100 nm.

Biomarkers (also referred to herein as “markers”) for preeclampsia can be derived from microparticles. Microparticles can be isolated from blood (e.g., serum or plasma) or other biological samples, by size exclusion chromatography. The elution buffer can be, for example, a buffered solution such as PBS, a non-buffered solution, water, or de-ionized water. The high molecular weight fraction can be collected to obtain a microparticle-enriched sample. Proteins within the microparticle-enriched sample are then extracted before digestion with a proteolytic enzyme such as trypsin to obtain a digested sample comprising a plurality of peptides. The digested sample is then subjected to a peptide purification/concentration step before analysis to obtain a proteomic profile of the sample, e.g., by liquid chromatography and mass spectrometry. In some embodiments, the purification/concentration step comprises reverse phase chromatography (e.g., ZIPTIP pipette tip with 0.2 μL C18 resin, from Millipore Corporation, Billerica, MA).

In certain embodiments, the microparticles are placental-derived exosomes or endothelial-derived exosomes. Such exosomes can be isolated using capture agents, such as antibodies, against surface biomarkers for these cells of origin. For example, placental-derived exosomes can be isolated using antibodies directed to PLAP (placental alkaline pjosphatase), Klotho, CD34, CD44 or leukemia inhibitory factor (LIF). Endothelial-derived exosomes can be isolated using antibodies directed to ICAM or VCAM.

Provided herein are compositions of matter comprising one or a plurality of preeclampsia biomarkers in substantially pure form. The biomarkers can be mixed in a container, or can be physically separated, for example, through attachment to solid supports at different addressable locations. As used herein, a chemical entity, such as a polynucleotide or polypeptide, is “substantially pure” if it is the predominant chemical entity of its kind in a composition. This includes the chemical entity representing more than 50%, more than 80%, more than 90% or more than 95% or of the chemical entities of its kind in the composition. A chemical entity is “essentially pure” if it represents more than 98%, more than 99%, more than 99.5%, more than 99.9%, or more than 99.99% of the chemical entities of its kind in the composition. Chemical entities which are essentially pure are also substantially pure.

### IV. Biomarker Discovery and Detection

### A. Analytical Methods

### 1. Statistical Analysis

A measurement of a variable, such as sequencing reads mapping to a position, can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, e.g., category into which the sequencing read is classified. Measurements of a variable on an ordinal scale produce a ranking, such as “first”, “second”, “third”. Measurements on a ratio scale include, for example, any measure on a pre-defined scale, absolute number of reads, normalized or estimated numbers, as well as statistical measurements such as frequency, mean, median, standard deviation, or quantile. Measurements that involve quantification are typically determined at the ratio scale level.

In some embodiments, analysis involves statistical analysis of a sufficiently large number of samples to provide statistically meaningful results. Such methods, or tools, include, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression, stepwise regression, ridge regression, lasso regression, elasticnet regression) or non-parametric analysis (e.g., Wilcoxon rank-sum test, Wilcoxon sign-rank test, sign test). In some embodiments, such tools are included in statistical packages such as MATLAB, JMP Statistical Software and SAS. Such methods produce models or classifiers which one can use to classify a particular biomarker profile into a particular state. In some embodiments, statistical analysis can be implemented by machine learning.

### 2. Machine Learning

In some embodiments, analysis can involve implementing machine learning techniques such as classification models including linear and non-linear models, e.g., processes such as CART—classification and regression trees), artificial neural networks such as back propagation networks, discriminant analyses (e.g., Bayesian classifier or Fischer analysis), logistic classifiers, and support vector classifiers (e.g., support vector machines).

Classification models, also referred to as models, can be generated by mathematical analysis, including by machine learning techniques that perform analysis of datasets of biomarker measurements derived from subjects classed into one or another group.

Diagnostic tests are characterized by sensitivity (percentage classified as positive that are true positives) and specificity (percentage classified as negative that are true negatives). The relative sensitivity and specificity of a diagnostic test can involve a trade-off—higher sensitivity can mean lower specificity, while higher specificity can mean lower sensitivity. These relative values can be displayed on a receiver operating characteristic (ROC) curve. The diagnostic power of a set of variables, such as biomarkers, is reflected by the area under the curve (AUC) of an ROC curve.

In some embodiments, the classifiers of this disclosure have a sensitivity, specificity, positive predictive value or negative predictive value of at least 85%, at least 90%, at least 95%, at least 98%, or at least 99%. Classifiers of this disclosure have an AUC of at least 0.6, at least 0.7, at least 0.8, at least 0.9 or at least 0.95.

Classification can be based on a measurement of a biomarker being above or below a selected cutoff level. In certain embodiments, a cutoff value is obtained by measuring biomarker levels in a plurality of positive and negative reference samples, e.g., at least 10, 20, 50, 100 or 200 samples of each type. A cutoff can be established with respect to a measure of central tendency, such as mean, median or mode in the negative samples. A measure of deviation from this measure of central tendency can be used to set the cutoff. For example, the cutoff can be set based on variance or standard deviation. For example, the cutoff can be based on Z score, that is, a number of standard deviations above a mean of normal samples, for example one standard deviation, two standard deviations, three standard deviations or four standard deviations. For example, cutoff values can be selected so that the diagnostic test has at least 80%, 90%, 95%, 98%, 99%, 99.5%, or 99.9% sensitivity, specificity and/or positive predictive value.

Numerically, an increased risk is associated with an odds ratio of over 1.0, preferably over 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 for preeclampsia.

In other embodiments, further provided herein is the measurement of biomarkers for pre-term birth from the same microparticle-enriched fraction used for measurement of preeclampsia biomarkers, and their use for predicting risk of preterm birth. Biomarkers for preterm birth are described, for example, in US publication 2015-0355188 (“Biomarkers for preterm birth”) and in International Application WO 2017/096405 (“Use of circulating microparticles to stratify risk of preterm birth”).

### B. Biomarkers

As used herein, the term “biomarker” refers to a biological molecule, the presence, form or amount of which exhibits a statistically significant difference between two states. Accordingly, biomarkers are useful, alone or in combination, for classifying a subject into one of a plurality of groups. Biomarkers may be naturally occurring or non-naturally occurring. For example, a biomarker may be a naturally occurring protein or a non-naturally occurring fragment of a protein. Fragments of a protein can function as a proxy or surrogate peptide for the protein or as stand-alone biomarkers.

Provided herein are protein biomarkers associated with increased risk of preeclampsia. Biomarkers for preeclampsia are presented in Table 2, Table 3 and Table 4.

Other biomarkers and panels of biomarkers for predicting increased risk of preeclampsia are described in International Publication WO 2019/152741, published Aug. 8, 2019, incorporated herein by reference in its entirety.

The biomarkers can be detected using de novo sequencing of proteins from microparticles isolated from a sample (e.g., blood) taken from a pregnant woman. Proteins can be sequenced by mass spectrometry, e.g., single or double (MS/MS) mass spectrometry. Both parent proteins and peptide fragments of parent proteins are useful as biomarkers of preeclampsia. Unless otherwise specified, a named protein biomarker encompasses detection by surrogate, e.g., fragments of the protein.

Proteins, e.g., peptides, detected by mass spectrometry are analyzed to identify those that are up-regulated (increased in amounts) or down-regulated (decreased in amounts) compared with controls. Proteins showing statistically significant differential expression are further analyzed to identify the parent protein. Such proteins can be identified in a protein database such as SwissProt.

In certain embodiments, biomarkers are analyzed as a panel comprising a plurality of the biomarkers. A panel can exist as a conceptual grouping, as a composition of matter (e.g., comprising purified biomarkers, or as an article, such as solid support attached to a capture reagent such as an antibody, further bound to the biomarker. The solid support can be, for example, one or more solid particles, such as beads, or a chip in which biomarkers are attached in an array format.

In certain embodiments, biomarkers can be comprised in a composition in which the peptide biomarker is paired with and a stable isotopic standard of the peptide. Such compositions are useful for detection in multiple reaction monitoring mass spectrometry.

For purposes of mass spectrometry, proteins can be detected intact, or through fragmentation, e.g., in multiple reaction monitoring (MRM). In such cases, proteins can be fragmented proteolytically before analysis. Proteolytic fragmentation includes both chemical and enzymatic fragmentation. Chemical fragmentation includes, for example, treatment with cyanogen bromide. Enzymatic fragmentation includes, for example, digestion with proteases such as trypsin, chymotrypsin, LysC, ArgC, GluC, LysN and AspN. Detection of these protein fragments, or fragmented forms of them produced in mass spectrometry, can function as surrogates for the full protein.

### 1. Biomarkers Identified from Bivariate Analysis

In some embodiments, bivariate statistical analysis of microparticle-associated proteins can identify the biomarkers of Table 2. Table 2 indicates the abbreviated name of the biomarker, the full name of the biomarker, the biologic function, the gene name encoding the protein and the adjusted p value of the biomarker.

Biomarkers used for predictions of preeclampsia requiring delivery in <=35 weeks gestation can be one or more than one biomarker selected from all of the biomarkers in Table 2, below. Such biomarkers show statistically significant difference in preeclampsia requiring delivery in <=35 weeks gestation versus normal birth in bi-variate analysis. Biomarkers selected may all be up-regulated, all be down-regulated or a combination of both up and down regulated biomarkers.

### 2. Biomarkers Identified With Machine Learning

Biomarkers identified in the previous machine learning operation were curated against the STRING protein database. Proteins either not included in the STRING database or identified as having fewer than four interactions with other proteins in the database were removed. The remaining proteins had a known biological function. Data relating to the remaining proteins was for the subject to machine learning.

Using machine learning on data produced by HRAM mass spectrometry analysis, other well-performing biomarkers were discovered, presented in Table 3. Table 3 shows the results of machine learning analysis of panels of proteins including four or more proteins. Using a cross-validation procedure applied over 100 iterations, the protein biomarkers occurring most frequently in the panels were identified. Proteins occurring at least five times in the hundred iterations are presented in Table 3.

Among the panels of biomarkers with high predictive value for PE requiring delivery in <=35 weeks gestation were the following: a panel of biomarkers comprising 2, 3 or 4 biomarkers selected from (1) C1RL, GP1BA, VTNC, and ZA2G, (2) C1RL, GP1BA, VTNC or (3) APOH, or GP1BA, VTNC, C1RL, ZA2G, APOC2, APOH, and JPH1.

### 3. Biomarker Clusters Associated with Preeclampsia

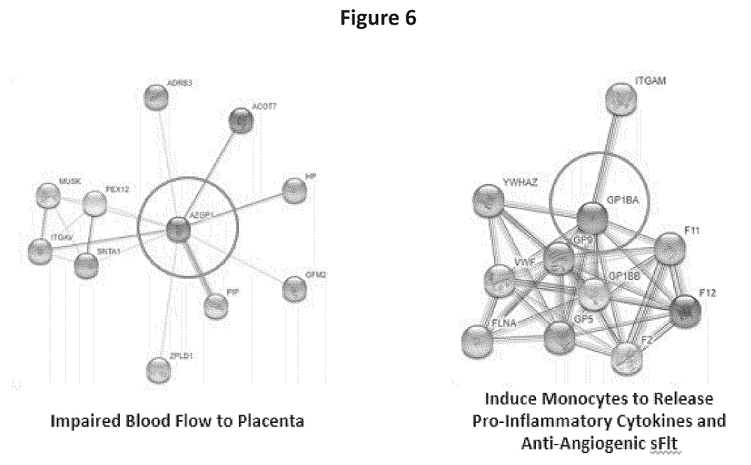

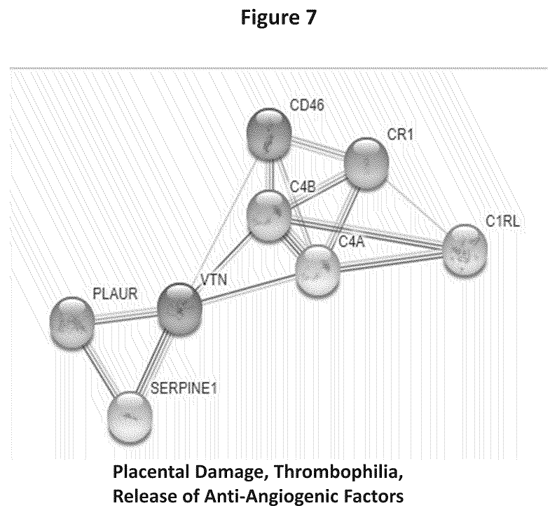

Table 4 shows the results of an unsupervised learning approach to subjects included in the preeclampsia requiring delivery in <=35 weeks gestation group only. Subjects could be classified into two associated clusters based on proteins differentially expressed (up-regulated or down-regulated) in each cluster. Associated cluster 1 was characterized by proteins associated with blood coagulation (FIG. 6). Associated cluster 2 was characterized by proteins associated with complement activity (FIG. 7). The implication of this differential protein expression in the two clusters implies a different therapeutic approach to women in each cluster. Accordingly, once a subject has been classified as being at increased risk of preeclampsia requiring delivery in <=35 weeks gestation, e.g., by the methods described herein, the subject can be further classified into one of two preeclampsia clusters: Coagulation-associated preeclampsia and complement-associated preeclampsia.

### C. Methods of Detection

Biomarkers can be detected and quantified by any method known in the art. This includes, without limitation, immunoassay, chromatography, mass spectrometry, electrophoresis and surface plasmon resonance.

Detection of a biomarker includes detection of an intact protein, or detection of surrogate for the protein, such as a fragment.

Immunoassay methods include, for example, radioimmunoassay, enzyme-linked immunosorbent assay (ELISA), sandwich assays and Western blot, immunoprecipitation, immunohistochemistry, immunofluorescence, antibody microarray, dot blotting, and FACS.

Chromatographic methods include, for example, affinity chromatography, ion exchange chromatography, size exclusion chromatography/gel filtration chromatography, hydrophobic interaction chromatography and reverse phase chromatography, including, e.g., HPLC.

### 1. Mass Spectrometry

In some embodiments, detecting the level (e.g., including detecting the presence) of a microparticle-associated protein is accomplished using a liquid chromatography/mass spectrometry (LCMS)-based proteomic analysis. In an exemplary embodiment the method involves subjecting a sample to size exclusion chromatography and collecting the high molecular weight fraction (e.g., by size-exclusion chromatography) to obtain a microparticle-enriched sample. The microparticle-enriched sample is then disrupted (using, for example, chaotropic agents, denaturing agents, reducing agents and/or alkylating agents) and the released contents subjected to proteolysis. The disrupted microsome preparation, containing a plurality of peptides, is then processed using the tandem column system described herein prior to peptide analysis by mass spectrometry, to provide a proteomic profile of the sample. The methods disclosed herein avoid the necessity of protein concentration/purification, buffer exchange and liquid chromatography steps associated with previous methods.

Proteins in a sample can be detected by mass spectrometry. Mass spectrometers typically include an ion source to ionize analytes, and one or more mass analyzers to determine mass. Mass analyzers can be used together in tandem mass spectrometers. Ionization methods include, among others, electrospray or laser desorption methods. Mass analyzers include quadrupoles, ion traps, time-of-flight instruments and magnetic or electric sector instruments. In certain embodiments, the mass spectrometer is a tandem mass spectrometer (e.g., “MS-MS”) that uses a first mass analyzer to select ions of a certain mass and a second mass analyzer to analyze the selected ions. One example of a tandem mass spectrometer is a triple quadrupole instrument, the first and third quadrupoles act as mass filters, and an intermediate quadrupole functions as a collision cell. Mass spectrometry also can be coupled with up-stream separation techniques, such as liquid chromatography or gas chromatography. So, for example, liquid chromatography coupled with tandem mass spectrometry can be referred to as “LC-MS-MS”.

Mass spectrometers useful for the analyses described herein include, without limitation, Altis™ quadrupole, Quantis™ quadrupole, Quantiva™ or Fortis™ triple quadrupole from ThermoFisher Scientific, the 8050 or 8060 triple quadruploes from Shimadzu, the Xevo TQ-XS™ triple quadrupole from Waters, QSight™ Triple Quad LC/MS/MS from Perkin Elmer, and others.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods and compositions disclosed herein. Suitable peptide MS and MS/MS techniques and systems are known in the art (see, e.g., Methods in Molecular Biology, vol. 146: “Mass Spectrometry of Proteins and Peptides”, by Chapman, ed., Humana Press 2000; Kassel & Biemann (1990) Anal. Chem. 62:1691-1695; Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more peptides. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS).

Selected reaction monitoring is a mass spectrometry method in which a first mass analyzer selects a protein of interest (precursor), a collision cell fragments the protein into product fragments and one or more of the fragments is detected in a second mass analyzer. The precursor and product ion pair is called an SRM “transition”. The method is typically performed in a triple quadrupole instrument. When multiple fragments of a protein are analyzed, the method is referred to as Multiple Reaction Monitoring Mass Spectrometry (“MRM-MS”).

Typically, protein samples are digested with a proteolytic enzyme, such as trypsin, to produce peptide fragments. Heavy isotope labeled analogues of certain of these peptides are synthesized as standards. These standards are referred to as Stable Isotopic Standards or “SIS”. SIS peptides are mixed with a protease-treated sample. The mixture is subjected to triple quadrupole mass spectrometry. Peptides corresponding to the daughter ions of the SIS standards and the target peptides are detected with high accuracy, in either the time domain or the mass domain. Usually, a plurality of the daughter ions is used to unambiguously identify the presence of a parent ion, and one of the daughter ions, usually the most abundant, is used for quantification. SIS peptides can be synthesized to order, or can be available as commercial kits from vendors such as, for example, e.g., ThermoFisher (Waltham, MA) or Biognosys (Zurich, Switzerland).

As used herein, the terms “multiple reaction monitoring (MRM)” or “selected reaction monitoring (SRM)” refer to a MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, the assay can include standards that correspond to the analytes of interest (e.g., peptides having the same amino acid sequence as that of analyte peptides), but differ by the inclusion of stable isotopes. Stable isotopic standards (SIS) can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Accordingly, detection of a protein target by MRM-MS involves detection of one or more peptide fragments of the protein, typically through detection of a stable isotope standard peptide against which the peptide fragment is compared. Typically, an SIS will, itself, be fragmented in a collision cell as the original digested fragment, and one or more of these fragments is detected by the mass spectrometer.

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5(4):573-588. Mass spectrometry-based assays can be advantageously combined with upstream peptide or protein separation or fractionation methods, such as, for example, with the tandem column system described herein.

### V. Methods of Assessing Risk of Preeclampsia Requiring Delivery in <=35 Weeks Gestation

The phrase “increased risk” of a condition, as used herein, indicates that a subject has a greater likelihood of developing the condition than a general population of subjects. So, for example, a subject who is at “increased risk of preeclampsia” has a greater likelihood of developing preeclampsia than a general population of subjects at the same stage of pregnancy, optionally compared with a population sharing one or more demographic or risk factors. The phrase “increased risk of preeclampsia requiring delivery in <=35 weeks gestation” as used herein indicates that a pregnant subject has a greater likelihood of developing preeclampsia requiring delivery in <=35 weeks gestation than a general population of subjects at the same stage of pregnancy, optionally compared with a population sharing one or more demographic or risk factors. The phrase “increased risk of blood coagulation-associated preeclampsia” or “increased risk of complement activity-related preeclampsia” as used herein indicates that a pregnant subject has a greater likelihood of developing each of these conditions compared with that of a general population of subjects at the same stage of pregnancy, optionally compared with a population sharing one or more demographic or risk factors. These may include, for example, age, status/result of prior pregnancy, hypertension, protein in urine, race/ethnicity, medical history, prior pregnancy history, smoking/drug history, and the like. For example, a test may indicate that a woman at 10-12 weeks of pregnancy has a higher risk of developing preeclampsia than a general or control population of woman at 10-12 weeks or pregnancy. In certain embodiments, the increased risk is distinguished from subject at increased risk of preeclampsia not requiring delivery in <=35 weeks gestation.

### A. Preeclampsia Requiring Delivery In <=35 Weeks Gestation

Provided herein are methods of assessing risk for preeclampsia, in particular, preeclampsia requiring delivery in <=35 weeks gestation, for example, classifying a pregnant human female as being at increased risk of preeclampsia requiring delivery in <=35 weeks gestation. The methods can involve determining a measure of one or a plurality of the biomarkers in Table 2 or Table 3, and correlating the measure to risk of preeclampsia requiring delivery in <=35 weeks gestation. For example, one can use a panel that includes 2, 3, 4, 5, 6 or more, or, no more than 2, 3, 4, 5, 6, biomarkers from the tables in the determination. In general, an amount of a biomarker that shows a difference compared to a control amount of the biomarker (e.g., a healthy pregnant female or a non-preeclampsia pregnant female), which difference is statistically significant, is associated with increased risk of preeclampsia requiring delivery in <=35 weeks gestation. The difference can be an up-regulation or a down-regulation, which can be easily determined by the practitioner. Alternatively, determination may be based on a classification algorithm that may employ non-linear and/or hyperdimensional methods.

In certain embodiments, the methods further comprise performing uterine artery Doppler ultrasound or measuring maternal blood pressure.

Methods of assessing risk of preeclampsia can involve classifying a subject as at increased risk of preeclampsia based on information including at least a measure of at least one biomarker of this disclosure.

Classifying can employ a classification model(s) and/or model(s) determined by statistical analysis and/or machine learning.

In exemplary embodiments provided herein is a method for assessing risk of preeclampsia requiring delivery in <=35 weeks gestation, in a pregnant subject, the method comprising: (a) preparing a microparticle-enriched fraction from a blood sample from the pregnant subject; (b) determining a measure of one or more microparticle-associated protein biomarkers in the fraction, wherein the one or more protein biomarkers are selected from a protein biomarker of Table 2, Table 3, Table 4, or the markers associated with the identified clusters 1 or 2, as respectively show in FIGS. 6 and 7; and (c) assessing the risk of preeclampsia requiring delivery at <=35 weeks gestation based on the measure.

In some embodiments the biomarkers are associated with coagulation activation (e.g. platelet activation). In exemplary embodiments, the biomarkers are associated with spiral artery remodeling. In exemplary embodiments, the biomarkers are associated with platelet aggregation, and/or monocyte activation. Exemplary biomarkers include those presented in FIG. 6. Exemplary biomarkers associated with spiral artery remodeling include ZA2G (e.g. a biomarker involved in the regulation of fibrosis). Exemplary biomarkers associated with platelet aggregation, and/or monocyte activation include GP1BA (e.g. a biomarker involved in the binding of platelets, inducing monocytes to release pro-inflammatory cytokines). These biomarkers may be involved in placental damage, thrombophilia, and/or release of anti-angiogenic factors, underlying potentially severe preeclampsia.

In some embodiments, the biomarkers are associated with complement activation. Exemplary biomarkers associated with complement activation include those presented in FIG. 7. Exemplary biomarkers include VTNC and C1RL. These biomarkers may be involved in placental damage, thrombophilia, and/or release of anti-angiogenic factors, underlying potentially severe preeclampsia.

### B. Subtypes of Preeclampsia

Further provided herein are methods of determining a subtype of preeclampsia of a pregnant subject already determined to be at increased risk of preeclampsia.

Unsupervised learning methods were used to analyze biomarkers in microparticles from blood taken at 10 to 12 weeks of pregnancy from subjects who developed preeclampsia requiring delivery in <=35 weeks gestation. This analysis identified two clusters of one subjects. A first cluster was characterized by an increased concentration of proteins involved in promoting blood coagulation, e.g. those associated with platelet activation (shown in FIG. 6). This cluster is referred to herein a “coagulation-associated preeclampsia.” A second cluster was characterized by an increased concentration of proteins involved in complement activation (shown in FIG. 7). This cluster is referred to herein a “complement-associated preeclampsia.” Exemplary biomarkers that characterize these subtypes are set forth in Table 4.

Methods of predicting whether a pregnant subject will develop coagulation-associated or complement-associated preeclampsia involve, first, establishing that the subject is at increased risk of developing preeclampsia and, second, predicting preeclampsia subtype based on their biomarker cluster profile. Subjects can be classified as being at increased risk of developing preeclampsia requiring delivery in <=35 weeks gestation by the methods described herein or any other available method. Once determined, the subject can be classified as to subtype using methods described herein.

### VI. Methods of Treating Subjects at Increased Risk of Preeclampsia

Methods of treating pregnant subjects suffering from or at increased risk of preeclampsia include administration of therapeutic interventions useful in treating preeclampsia. This includes, for example, administration of pharmaceutical drugs to treat elevated blood pressure, administration of drugs such as aspirin (e.g., low dose aspirin, e.g., 80 mg.), administration of statins and intensified monitoring for symptoms of preeclampsia. It also includes administration of targeted inhibitors of complement activation.

The particular intervention chosen can be tailored to the subtype of preeclampsia to which the subject belongs. Subjects belonging to the coagulation-associated preeclampsia subtype can be treated by administration of platelet aggregation inhibitors. These include, for example, aspirin, clopidogrel, cilostazol, prasugrel, ticagrelor, eculizumab and caplacizyumab. Subjects belonging to the complement-associated preeclampsia subtype can be treated by administration of complement activation inhibitors. These include, for example, an inhibitor of C1r/C1s, kallikrein, C1 inhibitor, serine protease inhibitor), a soluble complement regulator (e.g., factor I cofactor), an anti-complement antibody (e.g., anti-C5, anti-C5a, anti-CD20, anti-CD38), anti-Factor D, anti-factor B, anti-properdin), a complement component inhibitor, a receptor antagonist.

Interventions can be administered before the onset of symptoms, or when symptoms are present. Methods can involve delivering platelet aggregation inhibitors, but not (or significantly less amounts of) complement activation inhibitors to subjects in the coagulation-associated subtype, and delivering complement activation inhibitors but not (or significantly less amounts of) platelet aggregation inhibitors, to subjects in the complement activation subtype.

### VII. Kits and other Articles of Manufacture

In another embodiment, provided herein are articles of manufacture, e.g., kits of reagents useful in detecting in a sample biomarkers for increased risk of preeclampsia, in particular, preeclampsia requiring delivery in <=35 weeks gestation. Reagents capable of detecting protein biomarkers include but are not limited to antibodies. Antibodies capable of detecting protein biomarkers are also typically directly or indirectly linked to a molecule such as a fluorophore or an enzyme, which can catalyze a detectable reaction to indicate the binding of the reagents to their respective targets.

In some embodiments, the kits further comprise sample processing materials comprising a high molecular weight gel filtration composition (e.g., agarose such as SEPHAROSE) in a low volume (e.g., 1 ml, 3 ml, 5 ml, 10 ml) vertical column for rapid preparation of a microparticle-enriched sample from plasma. For instance, the microparticle-enriched sample can be prepared at the point of care before freezing and shipping to an analytical laboratory for further processing.

In some embodiments, the kits further comprise instructions for assessing risk of preeclampsia, in particular, preeclampsia requiring delivery in <=35 weeks gestation. As used herein, the term “instructions” refers to directions for using the reagents contained in the kit for detecting the presence (including determining the expression level) of a protein(s) of interest in a sample from a subject. The proteins of interest may comprise one or more biomarkers of preeclampsia. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination.

In another embodiment, a kit comprises a container containing one or a plurality of stable isotope standard (SIS) peptides corresponding to peptide biomarkers, e.g., peptides produced from protease (e.g., trypsin) digestion of biomarker proteins. In another embodiment, a majority or all of the SIS peptides correspond to the biomarker peptides. In another embodiment, the kit further comprises the biomarker peptides which the SIS peptides correspond.

In another embodiment, provided is a composition of matter that includes protein biomarkers of preeclampsia and, for a plurality of those biomarkers, a corresponding stable isotope standard peptide. This can be prepared by combining a sample comprising proteins isolated from microparticles, with stable isotope standard peptides.

### VIII. Systems

In some embodiments, systems for assessing and/or classifying preeclampsia can include at least one computing device. Some non-limiting examples of the computing device include computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iPhone®, Samsung Galaxy®, Google Pixel®, etc.), etc. In some embodiments, the computing device can comprise a processor and a memory. Alternatively, the computing device can be communicably coupled to a processor and a memory. The memory can receive measures of one or more biomarkers provided herein measured from a sample. The memory can include computer readable instructions which, when executed (e.g., executed by the processor), classify the sample as at risk of preeclampsia or not at risk of preeclampsia. In some embodiments, the memory may be stored in a memory device such as a random access memory (RAM), a memory buffer, a hard drive, an erasable programmable read-only memory (EPROM), an electrically erasable read-only memory (EEPROM), a read-only memory (ROM), Flash memory, and the like. In some emodiments, the memory may be stored on a cloud-based platform such as Amazon web services®.

The processor may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like. In some embodiments, the processor can execute one or more modules (e.g., modules in a software code and/or modules stored in a memory) to classify preeclampsia.

The computing device, processor, and/or memory can be operatively coupled to a computer network with the aid of a communications interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The system can include a first computer connected with a second computer through a communications network, such as, a high-speed transmission network including, without limitation, Digital Subscriber Line (DSL), Cable Modem, Fiber, Wireless, Satellite and, Broadband over Powerlines (BPL). Accordingly, results providing classification of a sample as at increased risk or as not at increased risk of preeclampsia can be transmitted from a transmitting computing device to a remote receiving computer, such as located at the office of a healthcare provider or to a mobile device, such as a smart phone.

### EXAMPLES

### A. Background

The present study has two parts. In the first part, CMP-associated proteins were sampled from maternal plasma at the end of the first trimester and tested to see if they differed in pregnancies complicated by preeclampsia when compared to those that remain normotensive. In the second part, among cases of preeclampsia, circulating CMP proteins were classified into different groups that correlate with maternal clinical characteristics. For both parts of this analysis, CMPs sampled were generally present within maternal circulation at a median of 12 weeks gestation. As such, the origins of these particles were combination of maternal and placental. Though the origin of these particles were not distinguished; the results demonstrated the use of CMP-associated proteins biomarkers of PE risk and classifiers of PE subtype.

### B. Results

Sample Composition and Characteristics. We initially selected and processed 25 cases and 50 controls for analysis. However, after the initial sample processing had been completed, access to detailed patient medical records identified one case with HIV that had been incorrectly classified as HIV negative; in an additional case, a patient was found to be on an immune-modifying medication for a chronic autoimmune condition. These two cases were excluded in an a priori fashion from the sample classification analysis. We thus analyzed 23 cases and 50 controls.

The characteristics of the sample set are presented in Table 1. Median maternal age, pre-pregnancy BMI, and race of the cases and controls did not differ significantly (p<0.05). Similarly, the percent nullipara, married, and tobacco use during pregnancy were also statistically similar in both groups. Consistent with the known risk factors for preeclampsia, the incidence of chronic hypertension and preeclampsia in a prior pregnancy was higher among the cases. The median gestational age at plasma sampling was similar but, consistent with the study design, the cases were delivered at a significantly earlier gestational age. Predictably the birth weight was greater for the controls but the birthweight Z-score for gestational age was similar in both groups. The ratio of male-to-female infants was higher, but not significant, among the preeclampsia cases.

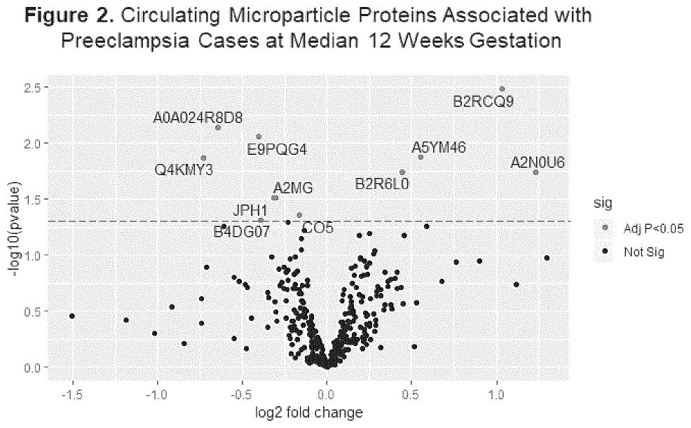

Signal processing using the Pinnacle Software (Optys Tech Corporation, Philadelphia, PA; see methods section) identified 226 proteins. These are presented in Supplement Table 4. Using a bivariate comparison for association with preeclampsia (cases) in the full dataset, the proteins were ranked by p-value adjusted for multiple comparisons (FIG. 2). Eleven proteins with an adjusted p-value<0.05 for comparison in case versus control with their associated biologic functions and gene names as documented in the UniProt database (www.uniprot.org, accessed Sep. 16, 2019) are listed in Table 2.

TABLE 1
Population characteristics

| | Preeclampsia | Controls | p- |
|---|---|---|---|
| Characteristic | (N = 23) | (N = 50) | valueª |
| | Median (IQR) | Median (IQR) | |
| | or N (%) | or N (%) | |
| Maternal age | 32.3 (28.1-36.6) | 31.3(26.2-36.9) | 0.63 |
| Maternal race | | | 0.74 |
| Black | 6 (26.0%) | 14 (28.0%) | |
| Hispanic | 2 (8.7%) | 10 (20.0%) | |
| White, non-Hispanic | 15 (65.2%) | 26 (52.0%) | |
| Nullipara | 5 (22.0%) | 14 (28.0%) | 0.42 |
| Smoking | 4 (17.4%) | 10 (20.0%) | 0.79 |
| Married | 16 (70.0%) | 31 (62.0%) | 0.89 |
| Chronic hypertension | 3 (13.0%) | 0 (0.0%) | 0.009 |
| Pre pregnancy BMI | 24.6 (21.1-29.1) | 25.5 (22.5-30.0) | 0.74 |
| PE in prior | 6 (33.3%) | 3 (8.3%) | 0.02 |
| pregnancy | | | |
| Prior spontaneous | 12 (66.7%) | 8 (22.2%) | 0.001 |
| PTB | | | |
| Gestational age at: | | | |
| sampling | 11.3 (8.7-13.4) | 11.1 (9.6-13.7) | 0.97 |
| delivery | 34.4 (32.0-<36) | 39.4 (38.9-40.4) | <0.001 |
| Birth metrics (in g): | | | |
| Birth weight | 2215 (1400-2811) | 3353 (3140-3725) | <0.001 |
| Z-score | −0.24 (−1.1-0.6) | −0.21 (−0.7-0.8) | 0.45 |
| Infant gender: | | | |
| female | 15 (65.2%) | 26 (52.0%) | 0.29 |
| male | 8 (34.8%) | 24 (48.0%) | |

^{a}p-values calculated with Wilcoxon Rank Sum test, ANOVA, Chi Square test, or Fisher Exact test where appropriate

TABLE 2
Bivariate comparison of differentially expressed proteins by
preeclampsia status

| | | Biologic | | Adjusted |
|---|---|---|---|---|
| Abbreviation | Name | Function | Gene | p-valueª |
| B2RCQ9^{b} | Highly similar to Homo | Unknown | N/A | 0.0033 |
| | sapiens heat shock | | | |
| | 70 kDa protein 1 | | | |
| A0A024R8D8^{b} | Progestogen- | Small molecule | PAEP | 0.0073 |
| | associated | binding | | |
| | endometrial protein | | | |
| E9PQG4^{b} | Myomegalin | Unknown | PDE4DIP | 0.0089 |
| A5YM46 | ERN2 protein | Protein kinase | REN2 | 0.013 |
| | | activity | | |
| Q4KMY3^{b} | C10orf28 protein | unknown | C10orf28 | 0.014 |
| B2R6L0 | Tubulin beta chain | cytoskeleton | N/A | 0.018 |
| | | structure | | |
| A2N0U6^{b} | VH6DJ protein | Immunoglobulin | VH6DJ | 0.018 |
| | | heavy chain | | |
| | | variable region | | |
| A2MG | Alpha-2- | Protease | A2M | 0.031 |
| | macroglobulin | inhibition | | |
| JPH1 | Junctophilin-1 | forms | JPH1 | 0.031 |
| | | junctional | | |
| | | membrane | | |
| | | complexes | | |
| CO5 | Complement C5 | Complement | C5 | 0.044 |
| | | activation | | |
| B4DG07 | Highly similar to | Unknown | N/A | 0.048 |
| | RAB6-interacting | | | |
| | protein 2 | | | |

^{a}P-values calculated with bivariate logistic regression. Bonferroni correction for multiple testing
^{b}Annotation unreviewed-protein information inferred

Table 2 indicates, given the unreviewed annotation and unclear function of several proteins, that there is a risk of persistent false discovery. Therefore, as described, we proceeded with the restricted dataset that included proteins with ≥4 documented functional associations (edges) as identified in the STRING database. This restricted the initial set of candidate proteins to those with prior annotation and known biologic interactions. Using the workflow described above, we sought panels of biomarkers associated with preeclampsia rather than a single analyte.

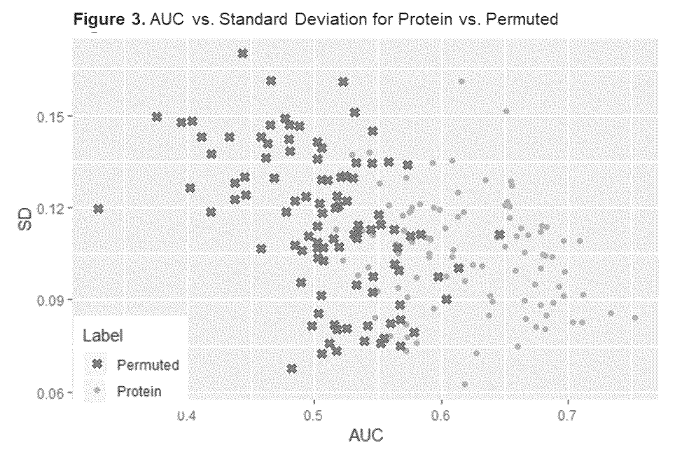

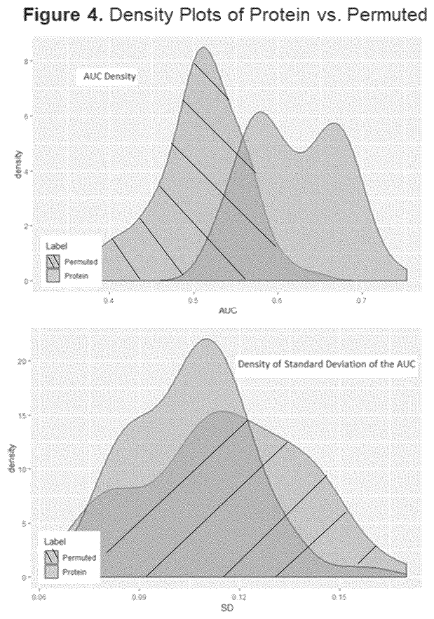

To further characterize the validity of the biomarker panel, we randomly permuted the sample labels (preeclampsia group versus control group) and re-applied the statistical workflow. The median and standard deviation of the outer AUC for the observed and permuted panels are compared in FIG. 3. The individual observed biomarkers have a clear tendency for higher mean AUC and a lower standard deviation of the AUC (right lower quadrant) when compared to the permuted samples. The median AUC for all the observed proteins in the panels was significantly greater than those in the permuted samples (0.62 vs. 0.51, p<0.0001). Consistent with its random origins, both the permuted AUC and the standard deviation of the permuted AUC have more central and normal-appearing distributions, whereas the identified proteins have asymmetric density plots (FIG. 4). The higher median AUC and the asymmetry in the density plots suggest that these biomarkers contain predictive information.

Table 3 lists the 17 individual proteins that reoccurred as a member of the multiple panels in at least five or more iterations. These proteins are ranked by the frequency with which they were members of the highest-scoring panel. These proteins were enriched in 26 biological processes mainly blood coagulation and hemostasis (N<5), restoration of injured tissue (N=6), and complement activation (N=4; all corrected p<0.05). Thirteen out of 17 proteins (76.5%) were enriched in the cellular compartments of extracellular exosomes and micro-vesicles (corrected p<0.001). Of these, 4 proteins (4/13=30.77%) demonstrated enrichment in placenta (corrected p=0.027; Supplemental Table 51).

TABLE 3
Most frequently recurrent individual proteins distinguishing
preeclampsia (delivery <= 35 weeks gestation) from controls in 100 multiplexed
panels

| Protein | Protein names | Biological function | Gene name | Frequency % |
|---|---|---|---|---|
| GP1BAª | Platelet glycoprotein Ib | blood coagulation | GP1BA | 79 |
| | alpha chain | | | |
| VTNC^{a} | Vitronectin | cell | VTN | 57 |
| | | adhesion/migration | | |
| C1RL^{a} | Complement C1r | complement activation | C1RL | 49 |
| ZA2G^{a} | Zinc-alpha-2-glycoprotein | cell adhesion | AZGP1 | 46 |
| APOC2 | Apolipoprotein C-II | cholesterol | APOC2 | 37 |
| | | homeostasis | | |
| APOH | Beta-2-glycoprotein 1 | blood coagulation | APOH | 30 |
| JPH1 | Junctophilin-1 | calcium ion transport | JPH1 | 28 |
| CO5 | Complement C5 | complement activation | C5 | 16 |
| HEP2 | Heparin cofactor 2 | blood coagulation | SERPIND1 | 16 |
| TPC11 | Trafficking protein | Golgi organization | TRAPPC11 | 14 |
| | complex subunit 11 | | | |
| MBL2 | Mannose-binding protein | complement activation | MBL2 | 11 |
| | C | | | |
| AACT | Alpha-1-antichymotrypsin | acute-phase response | SERPINA3 | 8 |
| DYH3 | Dynein heavy chain 3, | cilium-dependent cell | DNAH3 | 7 |
| | axonemal | motility | | |
| TSP1 | Thrombospondin-1 | cell adhesion | THBS1 | 7 |
| CAPS1 | Calcium-dependent | exocytosis | CADPS | 6 |
| | secretion activator 1 | | | |
| APOD | Apolipoprotein D | angiogenesis | APOD | 5 |
| LCAT | Phosphatidylcholine- | lipoproteins | LCAT | 5 |
| | sterol acyltransferase | metabolism | | |

Constituents of highest scoring panel

Among all combinations that distinguished normal delivery and preeclampsia requiring delivery in <=35 weeks, the panel of biomarkers with the highest mean AUC (0.79) and lowest mean standard deviation of the AUC (0.12) contained the following 4 biomarkers: complement C1r subcomponent protein (C1RL), platelet glycoprotein 1b alpha chain (GP1BA), vitronectin (VTNC), and zinc-alpha-2-glycoprotein (ZA2G) (Table 3). This combination of CMP-associated proteins occurred in 11% of the iterations. The next most frequent combination included a similar constituent set of analytes, but with beta-2-glycoprotein 1 (APOH) replacing ZA2G. (complement C1r subcomponent protein (C1RL), platelet glycoprotein 1 b alpha chain (GP1BA), vitronectin (VTNC), and beta-2-glycoprotein 1 (APOH).) This second combination occurred in 3% of the iterations.

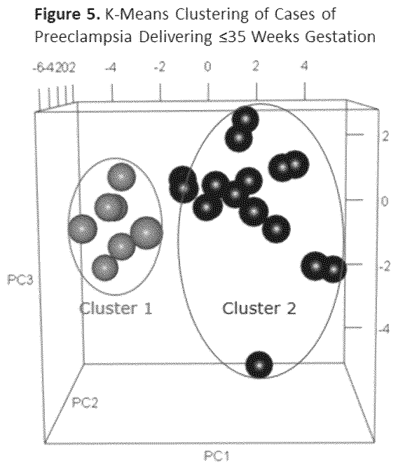

Clustering within the preeclampsia cases. To examine the potential for CMP-associated proteins to identify possible subgroups of disease among the subjects with preeclampsia, we performed unsupervised clustering using the K-means algorithm that was restricted to the cases in the prior analysis. Given the limited sample size, the candidate proteins for this clustering exercise were restricted to the 31 analytes that occurred in one or more of the iterated panels in the prior analysis. Preliminary silhouette analysis indicated that the optimal number of clusters for the restricted CMP protein data was two. The K-means procedure then identified a smaller cluster of 7 (Cluster 1) and a larger cluster of 16 (Cluster 2) and among the subjects with preeclampsia. FIG. 5 presents the two clusters projected on the first three principal components.

The Biological pathway enrichment analysis using the g:Profiler package indicated that the proteins associated with the cases in Cluster 1 displayed biological process enrichment in pathways related to platelet degranulation and intrinsic pathway of blood coagulation (N=3 and 2, p=0.02 and p=0.03, respectively. Molecular function enriched for protease binding and extracellular matrix structural constituents with multiple cellular components enriching for extracellular matrix and secretory granule function (all corrected p<0.05 The proteins associated with the cases in Cluster 2 were enriched in biological pathways for immune system responses, inflammatory and complement pathways (N=13, N=11, and N=10, respectively, all corrected p<0.05). Associated molecular functions tended to be centered on enzymatic activity. The cellular components associated with proteins in Cluster 2 related to blood microparticles, circulating microparticles and extracellular vesicles (N=11, 18 and 18, respectively, all corrected p<0.05). Proteins characteristic of subjects in Cluster 2 were also associated with biological pathways identified in g:Profiler as associated with tissue enrichment in placenta (N=8, p=0.049).

The 22 CMP-associated proteins with an unadjusted p-value>0.05 for differential expression in one of the two clusters are presented in Table 4 with their functional associations as annotated in the UniProt database. Four of the proteins had higher expression in Cluster 1, whereas 17 had higher expression in Cluster 2. After correction for multiple testing, vitronectin, pigment epithelium-derived factor, complement C4-A, and prothrombin retained significantly different expression between the two clusters.

TABLE 4
CMP associated proteins with differential expression between the clusters with protein functions

| | | | | Associated | | Bonferroni |
|---|---|---|---|---|---|---|
| Abbreviation | Name | Function | Gene | Cluster | p-value | Correction |
| IGJ | Immunoglobulin J | IgA/IgM binding | JCHAIN | 1 | 0.012 | 0.372 |
| | chain | | | | | |
| ZN251 | Zinc finger protein | RNA transcription | ZNF251 | 1 | 0.022 | 0.697 |
| | 251 | | | | | |
| ECM1 | Extracellular matrix | angiogenesis | ECM1 | 1 | 0.027 | 0.845 |
| | protein 1 | | | | | |
| CD5L | CD5 antigen-like | lipid synthesis | CD5L | 1 | 0.047 | 1.453 |
| | | regulator | | | | |
| A2MG | Alpha-2- | coagulation | A2M | 1 | 0.055 | 2.022 |
| | macroglobulin | | | | | |
| VTNC | Vitronectin | cell | VTN | 2 | 8.16E−06 | 0.0003 |
| | | adhesion/migration | | | | |
| PEDF | Pigment epithelium- | angiogenesis | SERPINF1 | 2 | 9.79E−05 | 0.003 |
| | derived factor | inhibitor | | | | |
| CO4A | Complement C4-A | complement | C4A | 2 | 0.0005 | 0.017 |
| | | activation | | | | |
| THRB | Prothrombin | blood coagulation | F2 | 2 | 0.001 | 0.033 |
| B4E1Z4 | Highly similar to | highly similar to | n/a | 2 | 0.004 | 0.109 |
| | Complement factor | Complement factor | | | | |
| | B | B | | | | |
| CO3 | Complement C3 | complement | C3 | 2 | 0.004 | 0.109 |
| | | activation | | | | |
| ANGT | Angiotensinogen | angiotensin- | AGT | 2 | 0.004 | 0.109 |
| | | activated signaling | | | | |
| | | pathway | | | | |
| CO2 | Complement C2 | complement | C2 | 2 | 0.005 | 0.142 |
| | | activation | | | | |
| PHLD | Phosphatidylinositol- | C-terminal protein | GPLD1 | 2 | 0.005 | 0.143 |
| | glycan-specific | lipidation | | | | |
| | phospholipase D | | | | | |
| FA12 | Coagulation factor | coagulation | F12 | 2 | 0.015 | 0.462 |
| | XII | | | | | |
| CFAH | Complement factor | complement | CFH | 2 | 0.018 | 0.569 |
| | H | modulation | | | | |
| CAPS1 | Calcium-dependent | vesicle exocytosis | CADPS | 2 | 0.025 | 0.777 |
| | secretion activator 1 | | | | | |
| CRP | C-reactive protein | acute-phase | CRP | 2 | 0.027 | 0.845 |
| | | response | | | | |
| KNG1 | Kininogen-1 | coagulation | KNG1 | 2 | 0.027 | 0.845 |
| HEP2 | Heparin cofactor 2 | coagulation | SERPIND1 | 2 | 0.033 | 1.020 |
| HEMO | Hemopexin | heme metabolic | HPX | 2 | 0.039 | 1.221 |
| PRG2 | Bone marrow | immune response | PRG2 | 2 | 0.047 | 1.453 |
| | proteoglycan | | | | | |

The population characteristics of the two clusters did not differ significantly (Supplement 3).

Supplement 3. Population Characteristics of Preeclampsia Cluster 1 and

| Characteristic | Cluster 1 | Cluster 2 | p-value^{a} |
|---|---|---|---|
| | (N = 7) | (N = 16) | |
| | Median (IQR) or N (%) | Median (IQR) or N (%) | |
| Maternal age | 28.6 (25-36.6) | 32.9 (29.4-36.7) | 0.59 |
| Maternal race | | | 0.39 |
| Black | 1 (14.3%) | 5 (31.3%) | |
| Hispanic | 0 (0.0%) | 2 (12.5%) | |
| White, non-Hispanic | 5 (71.4%) | 10 (62.5%) | |
| Nullipara | 1 (14.3%) | 4 (25.0%) | 0.57 |
| Smoking | 1 (14.3%) | 3 (19.0%) | 0.79 |
| Married | 6 (85.7%) | 10 (62.5%) | 0.27 |
| Chronic hypertension | 1 (14.3%) | 2 (12.5%) | 0.82 |
| Pre pregnancy BMI | 28.3 (24.7-30.8) | 23.6 (20.5-28.7) | 0.23 |
| PE in prior pregnancy | 2 (28.6%) | 4 (25.0%) | 0.71 |
| Prior spontaneous PTB | 4 (25.0%) | 8 (22.2%) | 0.89 |
| Gestational age at: | | | |
| sampling | 11.2 (8.5-13.7) | 11.4 (9.8-13.5) | 0.92 |
| delivery | 35.3 (33.8-35.4) | 34.1 (29.9-35.6) | 0.14 |
| Birth metrics (in g): | | | |
| Birth weight | 2339 (1956-2800) | 2168 (1181-2675) | 0.46 |
| Z-score | −0.41 (−1.3-0.5) | −0.20 (−0.9-0.6) | 0.53 |
| Infant gender: | | | |
| female | 4 (57.1%) | 11 (68.8%) | 0.59 |
| male | 3 (42.1%) | 5 (31.3%) | |

^{a}P-values calculated with Wilcoxon Rank Sum test, ANOVA, Chi Square test, or Fisher Exact test where appropriate

Tables 5 and 6 present the clinical characteristics of Clusters 1 and 2. The unadjusted median week of delivery was lower where the median maximum systolic and diastolic pressures, as well as the median 24-hour protein collection at diagnosis, are all significantly higher for Cluster 2 (Table 5). After correction for multiple testing, the week of delivery and urine protein remained significantly different. Table 6 presents other clinically relevant parameters in the setting of preeclampsia. Cluster 2 had significantly higher proportions of subjects with extreme analyte values for creatinine, fibrinogen, and plasma sodium levels in the unadjusted testing, these values do not remain significant after adjusting for multiple testing. However, the more severe analyte values in Table 6 are consistently associated with Cluster 2. Using a permutation algorithm, we can reject the null hypothesis that the observed is a purely random association with a single cluster (p<0.003).

TABLE 5
Differential pregnancy characteristics between clusters 1 and 2

| Cluster | Median Week | Median Maximum | Median Maximum | Median 24 hr |
|---|---|---|---|---|
| (N) | at Delivery | Systolic (mmHg) | Diastolic (mmHg) | Protein (gram) |
| 1 (N = 7) | 36.2 | 158 | 80 | 310 |
| 2 (N = 16) | 33.6 | 170 | 98 | 726 |
| P-value | 0.013 | 0.045 | 0.025 | 0.0026 |
| Bonferroni | 0.049 | 0.18 | 0.099 | 0.010 |
| Correction | | | | |

TABLE 6
Hematologic Differences Between Clusters 1 and 2

| | Creatinine | | Sodium | Uric | | | |
|---|---|---|---|---|---|---|---|
| | >1.0 | Fibrinogen > | <132 | Acid >5.7 | ALT >50 | AST >50 | Platelets |
| Cluster | mg/dL | 450 mg/dL | Mmol/L | mg/dL | U/L | U/L | <100,000 |
| (N) | N (%)^{a} | N (%)^{b} | N (%) | N (%) | N (%) | N (%) | N (%) |
| 1 (N = 7) | 0 (0.0%) | 0 (0.0%) | 0 (0.0%) | 3 (43.0%) | 1 (14.0%) | 1 (14.0%) | 1 (14.0%) |
| 2 (N = 16) | 8 (53.0%) | 6 (66.0%) | 8 (75.0%) | 8 (50.0%) | 2 (13.0%) | 2 (19.0%) | 4 (25.0%) |
| P-value | 0.02 | 0.02 | 0.02 | 0.08 | 0.91 | 0.80 | 0.57 |

^{a}Creatinine values not available on 1 subject in Cluster 2
^{b}Fibrinogen values not available on 6 subjects in Cluster 2

### C. Discussion

We have defined a set of CMP-associated proteins obtained between 10-12 weeks gestation that are able to stratify women at risk for preeclampsia, in particular, preeclampsia requiring delivery in <=35 weeks. We further identified two clinically-relevant clusters with differences in CMP-associated protein expression among the preeclampsia. Specifically, with regard to the former, we identified a panel of four CMP-associated proteins (C1RL, GP1BA, VTNC, ZA2G) that return clinically useful information for the prediction of preeclampsia. In the second portion of this analysis, we use CMP proteins in an unsupervised clustering analysis of preeclampsia cases to expose potentially diagnostically useful sub-structure among these cases of early-onset preeclampsia. The two clusters differ in their median gestational age at delivery, parameters of morbidity.

Characteristics of CMP-Associated Proteins as biomarkers of Preeclampsia at a Median of 12 Weeks: The proteins that distinguished preeclampsia requiring delivery in <=35 weeks gestation cases from healthy pregnancies were enriched in placenta and annotated to functional pathways including hemostasis, blood coagulation and restoration of injured tissue.

Clustering of Preeclampsia Subtypes: In the second portion of this analysis, we present evidence that CMP-associated proteins obtained in the first trimester indicate potential subgroups or sub-phenotypes among women who go on to develop preeclampsia. The unsupervised clustering of CMP-associated proteins into two groups demonstrated one group with a more clinically severe presentation associated with higher median 24-hour protein loss and higher median maximum systolic and diastolic blood pressures. These more severe preeclampsia-associated characteristics likely prompted the earlier median gestational age at delivery noted in this cluster. Likewise, other metrics of physiologic instability, including an increased likelihood of a creatinine greater than 1 mg/dL, fibrinogen greater than 450 mg/dL, plasma sodium less than 132 Mmol/L, and platelets less than 100,000 per ml, among other physiologic indicators, tended to be associated with this more severe cluster.

Characteristics of the Proteins Differentially Associated with Clusters 1 and 2: Among the proteins and their enriched biological pathways that differed between the two clusters, platelet and coagulation associated pathways were related to Cluster 1 where complement-associated proteins were associated with Cluster 2. Observations regarding CMP platelet degranulation and coagulation pathways associated with PE pathophysiology have been made previously. Multiple reports suggest that CMPs of platelet origin have been associated with PE. Activated platelets are involved in the remodeling of the spiral arteries and facilitation of cytotrophoblastic invasion at the end of the first trimester. CMP mediated accumulation of activated platelets in the placental bed have been associated with sterile trophoblastic inflammation. Cluster 1, therefore, may be more involved in preeclampsia pathology associated with aberrant platelet function at the end of the first trimester.

Cluster 2 appears to be associated with pathology linked to complement activity. Complement C2, C3, and C4-A, as well as complement factor H, represent 25% ( 4/16) of the CMP-associated proteins with higher expression in the second cluster, whereas complement associated-proteins were not present in Cluster 1. Even after controlling for multiple testing, complement C4-A remained significantly associated with Cluster 2. Proteins with functions associated with coagulation (prothrombin, coagulation factor XII, kininogen-1, and heparin cofactor 2) were also associated with this same cluster although only prothrombin remained significant after correction for multiple testing. There is precedent for complement involvement in preeclampsia pathophysiology. Other authors have noted an association between early-onset/severe preeclampsia and complement function. Our observations suggest that it may be possible to identify a sub-category of cases of preeclampsia with a dominant complement-associated phenotype at the end of the first trimester. This may offer future potential for screening and therapeutic interventions. There is precedent for such interventions, and several complement inhibitors are currently being evaluated in clinical trials.

### Conclusion

CMP-associated proteins, collected between 10-12 weeks of gestation, can be used to stratify the risk of later severe preeclampsia. For those that screen positive, clustering can be used to further characterize the putative subtype of severe preeclampsia. Eventually, risk stratification and disease phenotype characterizations such as this may be able to optimize prophylactic and therapeutic interventions.

### D. Methods

Samples Collection. We performed a nested case-control study selected from the prospectively collected LIFECODES pregnancy biobank at Brigham and Women's Hospital, Boston, MA. Patients were approached at their first prenatal visit (median 10.2 weeks gestation). Eligibility criteria included patients who were >18 years of age, initiated their prenatal care at <15 weeks of gestation, and planned on delivering at Brigham and Women's Hospital. After written informed consent was obtained, EDTA plasma samples were obtained, aliquoted, and stored at −80 degrees centigrade. The biobanking and research protocol were approved by the institutional review board at Brigham and Women's Hospital.

Pregnancy dating was confirmed by ultrasound at weeks gestation. If consistent with the last menstrual period (LMP) dating, the LMP was used to determine the due date. If not consistent, then the due date was set by the earliest available ultrasound ≤12 weeks gestation. Full-term birth was defined as ≥37 weeks of gestation. Maternal race was determined by self-identification. The medical record for each subject in the LIFECODES biobank is independently reviewed by two Maternal-Fetal Medicine faculty physicians. Complications and outcomes for each subject are coded using a structured coding tool. The codes from each reviewer are then compared then disagreement in either pregnancy outcome or complication is adjudicated by a review committee. The definition of preeclampsia used by the faculty reviewers is consistent with that of the 2013 Task Force on Hypertension in Pregnancy. In order to maximize clinical relevance and avoid the unintentional picking of “ideal” cases pf preeclampsia, the cases were chosen randomly (exclusive of the exclusion criteria noted below) from within our biobank. The cases of preeclampsia presented here represent cases of preeclampsia with severe features. Cases with evidence of hemolysis, and hence suggestive of HELLP syndrome, were excluded from this analysis.

We defined exclusion criteria as preexisting medical disorders (preexisting diabetes, current cancer diagnosis, HIV, and hepatitis), and ultrasonically-documented fetal anomalies. The analysis was restricted to singleton gestations. We defined cases as subjects diagnosed with preeclampsia requiring delivery in <=35 weeks gestation. Controls were defined as subjects delivering after 37 weeks gestation without evidence of any of the hypertensive disease of pregnancy (chronic hypertension, gestational hypertension or preeclampsia). Our final sample size consisted of 25 cases that were selected at random from among the 175 subjects with preeclampsia requiring delivery in <=35 weeks. Controls were randomly matched 2:1 from among the 2,342 subjects meeting the criteria defined above. Cases and controls were matched by maternal age (+/−2 years) and gestational age of sampling (+/−2 weeks).

CMP Enrichment. We utilized Size Exclusion Chromatography (SEC) for CMP isolation. Our methods have been detailed in our prior publications. SEC has been evaluated and reviewed favorably as an efficient means for microparticle isolation. Anonymized EDTA plasma samples identified only by a study number that was agnostic to case or control status were randomly assorted and shipped on dry ice to the David H. Murdock Research Institute (DHMRI, Kannapolis, NC) where CMP-associated protein enrichment was carried out by SEC and isocratically eluted using the NeXosome® Elution Reagent. This involved NeXosome® Isolation Columns manufactured by AmericanBio, Inc. (Canton, MA). Briefly, these columns were packed by AmericanBio with 2% Sepharose 2B (pore size 60-200 nm) from GE Healthcare Bio-Sciences Corp. (Marlborough, MA) to a total packed volume of 10 mL and delivered to DHMRI under ambient shipping conditions. Once received by DHMRI, the columns were stored at 2-8° C. until use. Prior to using the columns for CMP isolation, they were allowed to equilibrate to room temperature and subsequently washed with NeXosome® Elution Regent. EDTA plasma samples were thawed and 1.0 mL of plasma was applied and allowed to incorporate into the NeXosome® Isolation Column. The plasma samples were not filtered, diluted, or pretreated prior to application to the columns. Following the incorporation of the sample into the column, the NeXosome® Elution Reagent was added and 1.0 mL column fractions were collected. As previously published, the eluted fractions yielded two peaks. The CMPs were captured in the column void volume and resolved from the high abundant soluble protein peak. To minimize the potential for batch effects, the processing of individual samples was performed in random order. Each CMP-containing fraction (0.5 mL aliquots of each fraction) was pooled within each individual sample and a total protein measurement was performed, using the Pierce™ BCA Protein Assay Kit (ThermoFisher Scientific). An aliquot containing a total protein of 200 μg from each individual CMP isolate pool was then transferred to 2-mL microcentrifuge tubes (VWR, Radnor, PA) and stored at −80° C. pending completion of all CMP isolate processing. All CMP isolates were then shipped on dry ice to ThermoFisher Scientific's Biomarker Research Initiatives in Mass Spectrometry (BRIMS) Center (Cambridge, MA) for proteomic analysis.

Liquid Chromatography-Mass Spectrometry. Study samples were sent on dry ice to the BRIMS institute. Additional aliquots were created and analyzed for system suitability and quality control analysis. These included PRTC standards for system suitability, pooled CMP digests for quality control analysis, and twelve peptide fractions for spectral library creation. Data was acquired via a Vanquish Horizon UHPLC coupled to a Fusion Lumos Orbitrap (ThermoFisher Scientific, Cambridge, MA) mass spectrometer. The spectral library was created on PD 2.1 and searched against Uniprot Human data base. Static cys carboxymethylation, differential deamidation, oxidation, and global proteome profiling were examined. Data were normalized based on TIC.

Briefly, for each sample, a total of 200 μg of CMP protein fraction pool was lyophilized and then dissolved and denatured/reduced with 50 μL 8 M guanidine hydrochloride, 250 mM Tris-HCl, 2.5% n-propanol, 10 mM DTT, pH 8.6 for 1 hour at 26° C. Alkylation of cysteines was performed by adding 4.5 μL of 500 mM sodium iodoacetate and allowing the sample to sit for 2 hours. Digestion buffer, 850 μL, 50 mM Tris-HCl 5 mM CaCl₂, 1% n-propanol, 0.35 mM DTT was added to each tube, and the samples transferred to a 2 mL 96 well ThermoFisher Scientific Polypropylene plate. Pierce TPCK modified trypsin was dissolved in 25 mM acetic acid to a final concentration of 22 mg/mL. To each well was added 250 μL of this trypsin solution and the plates where then sealed with a ThermoFisher Scientific Easypeel heat sealing foil. Samples were incubated with shaking at 37° C. for 16 hours. Post-digestion, 50 μL of acetic acid was added to each well and the samples lyophilized to dryness for 16 hours at 35 mTorr. Each sample was brought up in 450 μL of 2% methanol 1% acetic acid, containing 22.2 fmol/μL of the PRTC peptide mix. An aliquot of each sample 65 μl was transferred to another smaller ThermoFisherScientific plate (AB-1300) and sealed with a ThermoFisher Scientific Easypeel heat sealing foil and refrigerated at 4° C. until use.

Liquid Chromatography. Identical liquid chromatography methods were used for all methods, both library and individual sample quantification runs. Sample plates were loaded onto the UHPLC. The UHPLC system was plumbed with a column compartment divert valve, and a system divert valve. The Vanquish Active solvent preheater at 80° C. is connected to a 2.1×50 mm PS-DVB trap column containing 3 μm particles; the column compartment divert valve is then linked to two Acclaim RSLC 120 C18 2.2 μm analytical columns connected in tandem—the compartment housing the analytical columns is held at 60° C.—the analytical columns are, in turn, connected to the system divert valve. Solvent A on the system is Optima LC-MS grade water with 2% methanol and 0.2% formic acid. Solvent B is Optima LC-MS grade water:isopropanol:acetonitrile:formic acid, at a 10:10:80:0.2% ratio. From each well, 45 μL of sample is injected at 1 mL/min, the intra Trap-Analytical column divert column is kept diverted for 1 min to load and desalt the sample, the flow rate is dropped to 250 μL a min and the divert valve switched to the analytical column, and then a gradient of 10-38% B is applied over 50 minutes, after which the valve is set to divert again, and a 45 μL injection of formic acid is injected while ramping the flow and gradient to 1 mL/min and 100% B. Post-rinsing, the column is equilibrated to A for 6 min at 800 μL/min.

Library fractionation. Spectral libraries were made by taking 20% of every sample and making a large pool. This pool (2 mg) was injected onto a 4.6×250 mm PS-DVB column running a gradient of A 0.2% ammonium hydroxide, 50mM ammonium formate to B 0.2% ammonium hydroxide. Peptides are fractionated into a 12, 2 mL fractions in a 2.2 mL 96-well plate containing 100 μL of 50% acetic acid. Fractions are frozen/lyophilized and dissolved into 250 μL of 2% methanol, 1% acetic acid containing 22.2 fmol/μL of the PRTC peptide mix.

Mass Spectrometry. The Orbitrap was used to generate the peptide spectral libraries and run the individual test samples. Source settings on the system were set to 4.5 kV with a sheath of 25 units, aux of 5 units, and a sweep of 2 units with an ion capillary temperature of 325 and a gas temperature of 275 degrees C. The source housing drain was removed for this experiment. The Lumos was run in a data-dependent acquisition mode with a “cycle time” limit of 2 s, a full scan mass range of 350-1500 m/z with polysiloxane lock masses being used in the full scan only. For Library acquisition, stepped collision energy ±5% was used to give better peptide backbone coverage, with a cycle time of 2 s. For the data acquisition on the panel samples, a wide data-dependent acquisition scheme was used. For this setting, a 5-dalton isolation window was used with a 1.2 s cycle time to preserve full scan quantification integrity.

Database searching and data processing. Raw library files were searched against the IPI human database using SEQUEST with Proteome Discoverer version 2.2. Static carboxymethyl cysteines and dynamic oxidized methionines were used with a parent mass tolerance of 10 ppm and a product ion tolerance of 0.02 Da. Peptides were scored with Percolator and peptides with an FDR of 0.01 or less were used for the libraries.

Signal processing and initial data analysis were carried out using Pinnacle software for multiplexed MRM data analysis (version 1.0.92.0; Optys Tech Corporation). A spectral library was created by importing the Proteome Discoverer MSF files. For global proteome profiling, data were normalized based on total ion chromatograph. Quantified peptides were associated with the protein of origin based on a stratification that included an MSMS dot product score ≥0.6. Peptide library retention time tolerance was set to 1 min. The coefficient of variation within each group (cases versus controls) was required to be ≤30% and the false discovery rate of the spectral library was ≤0.05. Protein concentrations were calculated after normalization to the reference group's median value. Putative protein targets were identified and ranked by minimizing the coefficient of variation within group and the p-value associated with the difference between cases and controls. Normalized file areas were then exported for analysis.

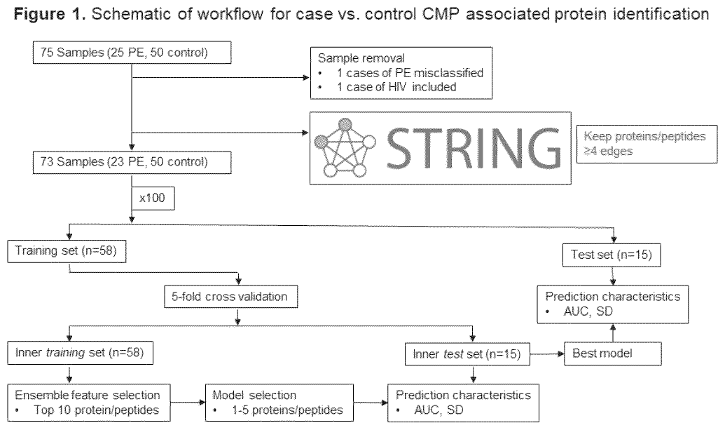

Sample Classification. We initially explored bivariate associations between preeclampsia and individual CMP-associated proteins with significance levels adjusted for multiple testing using the Bonferroni correction. We chose this correction given its conservative tendency. We then sought to establish a panel of CMP-associated proteins that would serve as a classifier for the risk of preeclampsia ≤35 weeks. Under the hypothesis that highly correlated analytes are likely to be members of the same biological pathways, we selectively excluded analytes with correlations ≥0.7. We adjudicated between two correlated analytes by retaining analytes with a greater number of overall correlations. By retaining the analyte with the greater number of correlations we are assuming that it is member in a larger number of pathways and hence more functionally important. To further enrich the remaining set of analytes to those with known systematic interactions, we adapted a principle from systems biology that highly interactive elements are more likely to correlate with the state of the overall system and screened the identified proteins for known direct interactions in the STRING database (FIG. 1; www.string-db.org, accessed May 25, 2019. Proteins with four or more edges were retained in the validation procedure. This step aided in the elimination of protein fragments (e.g. hypervariable regions) that represent potential false-positive findings. We termed the data prior to this step the “full” dataset, and that after this step the “restricted” dataset.

To select our putative panel, we chose a cross-validation procedure using logistic regression. As our intention was to create generalizable models and given the limitations of our sample size, the cross-validation procedure was applied over 100 iterations. For each iteration, the sample was randomly divided into a training and validation set (80 vs. 20 percent). The proteins in the training set were then ranked by their Akaike information criterion (AIC) using an ensemble feature selection procedure that examined the association between each protein and the outcome using a panel of bivariate tests. The top 10 individual proteins in each iteration were selected. The training set was subjected to 5-fold cross-validation. Accordingly, the training set was divided into 5 subsets of equal size. The elements of each subset were unique, and a sample could belong to only one subset per iteration. Four of the subsets were then combined and used to train a logistic regression on the fifth set. This was repeated five times such that each set functioned in the test role only once.

The glmulti package was used to evaluate all sets of predictors and rank each model by AIC. To avoid overfitting, given the limited sample size, the model was restricted to no more than 4 predictors. The model with the greatest area under the curve (AUC) and the lowest standard deviation of the AUC was then tested against the set-aside, external validation set. The AUC and standard deviation of the AUC of this external validation set was then recorded for the 100 iterations. The models were then ranked according to these parameters. The ranked models and the frequency with which individual proteins occurred overall models were recorded.

To confirm the utility of the workflow described above, we randomly permuted the case vs. control labels of the samples. The workflow was re-run as described above. Predictive statistics for the observed versus permuted data were then graphically compared (FIG. 1). The logic of this permutation was adapted from that of Yoffe et al. Early Detection of Preeclampsia Using Circulating Small non-coding RNA. Sci. Rep. (2018) doi:10.1038/s41598-018-21604-6.

Preeclampsia Sub-Classification. To explore potential sub-phenotypes within preeclampsia, we performed an unsupervised clustering analysis only among the cases from the restricted dataset. We used the Average Silhouette method to determine the appropriate number of clusters within the preeclampsia cases. K-means clustering was then used to partition the cases into the appropriate number of clusters. Both the Average Silhouette method and K-means were run using Euclidean space.

All statistical analyses were performed in the R computing environment (version 3.2.5; http:// ww.r-project.org). To obtain biological insight into the functional role of the identified proteins, we used g:Profiler package in R for the pathway enrichment analysis, tissue enrichment and identification of the associated Gene Ontology (GO) terms. Functional information on individual proteins was obtained from the UniProt database.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

1. A method for determining a subtype of preeclampsia in a pregnant subject, the method comprising:
(a) preparing a microparticle-enriched fraction from a blood sample from a pregnant subject at increased risk of preeclampsia;
(b) determining a measure of a panel of microparticle-associated protein biomarkers in the fraction, wherein the panel includes at least one coagulation-related biomarker and/or at least one complement activity-related biomarker; and
(c) assessing the form of preeclampsia based on the measure, wherein:
altered expression of a coagulation-related biomarker indicates blood coagulation-associated preeclampsia; and altered expression of a complement activity-related biomarker indicates complement-type preeclampsia.

2. The method of claim 1, further comprising determining that the subject is at increased risk of preeclampsia:
(a) determining a measure of one or more microparticle-associated protein biomarkers in a microparticle-enriched fraction from the subject, wherein the one or more protein biomarkers are associated with increased risk of preeclampsia; and
(c) determining an increased risk of the risk of preeclampsia, based on the measure or measures.

3. (canceled)

4. The method of claim 1, wherein the panel comprises (1) complement C1r subcomponent protein (C1RL), platelet glycoprotein 1b alpha chain (GP1BA), vitronectin (VTNC), and zinc-alpha-2-glycoprotein (ZA2G) or (2) complement C1r subcomponent protein (C1RL), platelet glycoprotein 1b alpha chain (GP1BA), vitronectin (VTNC), and beta-2-glycoprotein 1 (APOH).

5. The method of claim 1, 2, or 3, wherein:
(i) the at least one coagulation-related biomarker is selected from the group consisting of Immunoglobulin J chain, Zinc finger protein 251, Extracellular matrix protein 1, CD5 antigen-like, and Alpha-2-macroglobulin; and
(b) the at least one complement activity-related biomarkers is selected from the group consisting of Vitronectin, Pigment epithelium-derived factor, Complement C4-A, Prothrombin, Highly similar to Complement factor B, Complement C3, Angiotensinogen, Complement C2, Phosphatidylinositol-glycan-specific phospholipase D, Coagulation factor XII, Complement factor H, Calcium-dependent secretion activator 1, C-reactive protein, Kininogen-1, Heparin cofactor 2, Hemopexin, and Bone marrow proteoglycan.

6. The method of claim 1, wherein the altered expression associated with coagulation-associated preeclampsia is an increased expression compared with normal, and/or the altered expression associated with complement activity-associated preeclampsia is an increased expression compared with normal.

7. A method of treating a subtype of preeclampsia in a pregnant subject comprising:
(a) determining whether the subject is at increased risk of coagulation-associated preeclampsia or complement activity-type preeclampsia, wherein determining comprises associating a measure of one or more protein coagulation-related biomarkers or one or more complement activity-related biomarkers with coagulation-associated preeclampsia or complement activity-type preeclampsia; and
(b) treating the subject as follows:
(1) if the subject is determined to be at increased risk of coagulation-associated preeclampsia, administering to the subject a therapeutic intervention to decrease abnormal blood coagulation; or
(2) if the subject is determined to be at increased risk of complement activity-type preeclampsia, administering to the subject a therapeutic intervention to decrease abnormal complement activity.

8. The method of claim 7, comprising, before determining the type of preeclampsia, determining that the subject is at increased risk for preeclampsia.

9. The method of claim 7, wherein the therapeutic intervention for coagulation-associated preeclampsia, comprises administration of a platelet aggregation inhibitor, optionally wherein the platelet aggregation inhibitor is selected from the group consisting of aspirin, clopidogrel, cilostazol, prasugrel, ticagrelor, caplacizyumab.

10. The method of claim 7, wherein the therapeutic intervention for complement activity-type type preeclampsia, comprises administration of a pharmaceutical selected from the group consisting of a protease inhibitor, a soluble complement regulator, an anti-complement antibody, a complement component inhibitor, and a receptor antagonist.

11. A method for assessing risk of preeclampsia requiring delivery in <=35 weeks gestation, in a pregnant subject, the method comprising:
(a) preparing a microparticle-enriched fraction from a blood sample from the pregnant subject;
(b) determining a measure of one or more a panel of microparticle-associated protein biomarkers in the fraction, wherein the panel comprises one or more protein biomarkers arc selected from:
(i) a protein biomarker of Table 2; and
(ii) a protein biomarker of Table 3; and
(c) assessing the risk of preeclampsia requiring delivery at <=35 weeks gestation based on the measure.

12. The method of claim 11, wherein assessing the risk comprises distinguishing risk of preeclampsia requiring delivery at <=35 weeks gestation and preeclampsia not requiring delivery at <=35 weeks gestation.

13. The method of claim 11, wherein an increased amount of an up-regulated biomarker or a decreased amount of a down-regulated biomarker indicates increased risk of preeclampsia requiring delivery in <=35 weeks gestation.

14.-15. (canceled)

16. The method of claim 11, wherein the panel comprises a plurality of biomarkers selected from GP1BA, VTNC, C1RL, ZA2G, APOC2, APOH, and JPH1.

17. The method of claim 11, wherein the panel comprises complement C1r subcomponent protein (C1RL), platelet glycoprotein 1b alpha chain (GP1BA), vitronectin (VTNC), and zinc-alpha-2-glycoprotein (ZA2G).

18. The method of claim 11, wherein the panel comprises complement C1r subcomponent protein (C1RL), platelet glycoprotein 1b alpha chain (GP1BA), vitronectin (VTNC), and beta-2-glycoprotein 1 (APOH).

19. The method of claim, wherein the panel comprises no more than any of 10, 9, 8, 7, 6, 5, or 4 protein biomarkers.

20. The method of claim 11, wherein the sample is taken from the pregnant subject during the first trimester or second trimester of pregnancy.

21. The method of claim 20, wherein the sample is taken from the pregnant subject during weeks 10-12 of gestation.

22. The method of claim 11, wherein the pregnant subject is primigravida, multigravida, primiparous or multiparous.

23. The method of claim 11, wherein the pregnant subject has a singleton pregnancy or multiple pregnancy.

24. The method of claim 11, wherein the pregnant subject:
(a) is asymptomatic for preeclampsia;
(b) has no history of preeclampsia;
(c) has no other risk factors for preeclampsia; or
(d) has chronic hypertension.

25.-27. (canceled)

28. The method of claim 11, wherein the blood sample is plasma or serum.

29. The method of claim 11, wherein the microparticle-enriched fraction is prepared using size-exclusion chromatography.

30.-33. (canceled)

34. The method of claim 11, wherein the microparticles are further purified to enrich for placental-derived exosomes or vascular endothelial-derived exosomes.

35. The method of claim 11, wherein determining a measure comprises:
(a) mass spectrometry;
(b) liquid chromatography/mass spectrometry (LC/MS);
(c) liquid chromatography/triple quadrupole mass spectrometry; or
(d) multiple reaction monitoring.

36.-40. (canceled)

41. The method of claim 35, wherein determining the measure comprises determining a measure of a surrogate peptide of the protein biomarker.

42.-46. (canceled)

47. The method of claim 11, wherein the assessing comprises executing a classification rule, which rule classifies the subject as being at increased risk of preeclampsia requiring delivery in <=±weeks gestation, as being at increased risk of blood coagulation-associated preeclampsia and/or as being at increased risk of complement-type preeclampsia, and wherein execution of the classification rule produces a correlation between preeclampsia requiring delivery in <=±weeks gestation or term birth with a p value of less than at least 0.05.

48. The method of claim 1, wherein the assessing comprises executing a classification rule, which rule classifies the subject at being at risk of preeclampsia, and wherein execution of the classification rule produces a receiver operating characteristic (ROC) curve, wherein the ROC curve has an area under the curve (AUC) of at least 0.6, at least 0.7, at least 0.8 or at least 0.9.

49. The method of claim 1, wherein values on which the classification rule classifies a subject further include at least one of: maternal age, maternal body mass index, primiparous, and smoking during pregnancy.

50. The method of claim 11, wherein the classification rule employs cut-off, linear regression (e.g., multiple linear regression (MLR), partial least squares (PLS) regression, principal components regression (PCR)), binary decision trees (e.g., recursive partitioning processes such as CART - classification and regression trees), artificial neural networks such as back propagation networks, discriminant analyses (e.g., Bayesian classifier or Fischer analysis), logistic classifiers, and support vector classifiers (e.g., support vector machines).

51.-66. (canceled)

67. A computer-implemented method for generating a model to assess a risk of preeclampsia, the computer-implemented method comprising:
obtaining a dataset, the dataset comprising measurements associated with a plurality of biomarkers derived from each of a plurality of subjects; and
implementing a machine learning analysis to associate a set of biomarkers within the plurality of biomarkers with preeclampsia, wherein implementing the machine learning analysis generates a model to assess the risk of preeclampsia, wherein the risk of preeclampsia is one or more of risk of preeclampsia requiring delivery in <=±weeks gestation, risk of blood coagulation-associated preeclampsia and risk of complement-activity related preeclampsia, wherein the set of biomarkers are selected from the biomarkers of Table 2, Table 3, and Table 4.

68.-71. (canceled)

72. A computer-implemented method of assessing a risk of preeclampsia in a subject, the computer-implemented method comprising:
determining a quantitative measure of at least one biomarker in a sample; and
executing a classification rule based on the quantitative measure,
wherein the execution of the classification rule assesses the risk of preeclampsia in the subject (e.g. risk of preeclampsia requiring delivery in <=±weeks gestation, risk of blood coagulation-associated preeclampsia and/or risk of complement-activity related preeclampsia), and
wherein the classification rule implements at least one of cut-offs, linear regression, binary decision trees, artificial neural networks, discriminant analyses, logistic classifiers, and support vector classifiers,
wherein the at least one biomarker is selected from the biomarkers of Table 2, Table 3, and Table 4.

73.-75. (canceled)
